(19) United States
(12) Patent Application Publication
(10) Pub. No.: US 2026/0151277 A1
(43) Pub. Date: Jun. 4, 2026

(54) LOW CALIPER ABSORBENT ARTICLE WITH LOFTY TOPSHEET

(71) Applicant: THE PROCTER & GAMBLE COMPANY, Cincinnati, OH (US)

(72) Inventors: Amit Kumar KAUSHIK, West Chester, OH (US); Jingyi CHEN, Beijing (CN); Levente KISS, Budapest (HU)

(21) Appl. No.: 19/405,658

(22) Filed: Dec. 2, 2025

(30) Foreign Application Priority Data
Dec. 4, 2024 (WO) PCT/CN2024/136813

(51) Int. Cl. A61F 13/539; A61F 13/15; A61F 13/472; A61F 13/511; A61F 13/514; A61F 13/53

(52) U.S. Cl. CPC A61F 13/539; A61F 13/15707; A61F 13/472; A61F 13/5116; A61F 13/51456; A61F 2013/530802; A61F 2013/53908

(57) ABSTRACT

Example configurations disclosed herein are directed to an absorbent article including a liquid permeable topsheet, a liquid impervious backsheet, and an absorbent layer positioned therebetween. The topsheet includes a fibrous nonwoven material, an upper topsheet surface and a lower topsheet surface, and a first topsheet portion adjacent the upper topsheet surface and a second topsheet portion adjacent the lower topsheet surface. The absorbent layer includes a layer of open-cell foam having an upper foam surface that is in direct facing contact with the lower topsheet surface. The upper topsheet surface includes structured and unstructured regions. One or more unstructured regions of the upper topsheet surface exhibit a Sdcₐ of from 530 μm to 1000 μm when the topsheet is bonded to the absorbent layer with an adhesive. A portion of the adhesive penetrates into the second topsheet portion and the first topsheet portion is substantially free of adhesive.

### TECHNICAL FIELD

The present disclosure relates to absorbent articles, and more specifically to low caliper absorbent articles with lofty topsheets.

### BACKGROUND

Some segments of the consumer market for absorbent articles such as feminine hygiene pads prefer products with textured topsheets. In certain circumstances, an appropriately textured topsheet combined with other suitable product features can serve to reduce or desirably channel or control planar fluid migration, and facilitate fluid movement downwardly into the product structure, to underlying absorbent layers. Further, a suitably-textured topsheet can feel more comfortable against the skin, and provide enhanced breathability, for enhanced feelings of softness, coolness, and/or dryness. For these reasons, assemblies or subassemblies of absorbent articles that include topsheets together with conventional absorbent materials (i.e., cellulosic fiber or pulp, etc.), are often embossed to impart three-dimensional (3D) texture to the wearer-facing surface.

In a more modern configuration, relatively thin, low bulk, but still effectively absorbent feminine hygiene pads having an absorbent layer formed of open-cell foam have been marketed. Examples include particular offerings of ALWAYS brand and Liquid Pad brand pads manufactured and sold by The Procter & Gamble Company. These products are appreciated by some market segments because they have good absorption performance, while being relatively thin (i.e., relatively low-caliper) and quite resilient and pliable, making them comfortable to wear/use, and making them relatively discreet under outer clothing. Such pads are currently offered in a configuration having a topsheet formed of a relatively thin and untextured, low-basis weight nonwoven web material.

It has been learned that, for an absorbent system of a feminine hygiene pad to function effectively, it must be able to move fluid rapidly down through the topsheet to the absorbent structure beneath. A topsheet formed of fibrous nonwoven material wicks discharged fluid along surfaces of its fiber components, and through the inter-fiber passageways among the fibrous matrix. Thus, a substantial gap or air space between a topsheet and an underlying absorbent layer will hinder movement/transfer of fluid from the topsheet to the absorbent layer.

Accordingly, opportunity remains for development of an absorbent pad having a combination of features including those associated with current foam-based pads that are appreciated by consumer market segments, while having a relatively highly textured topsheet that provides for enhanced consumer perception, in a combination that does not compromise rapid fluid acquisition.

One issue that can arise when a textured topsheet is bonded to an absorbent layer that includes open-cell foam is that the textured topsheet may become unevenly adhered to the absorbent layer. This issue of uneven bonding can be exasperated when the absorbent layer has a variable thickness. When the textured topsheet is unevenly adhered to the absorbent layer, the topsheet may exhibit textural irregularities, i.e., the topsheet may appear to provide extra cushioning in some areas while other areas may appear to provide only minimal cushioning. Consumers may interpret these textural irregularities to mean that the absorbent article is of inferior quality, or that the absorbent article will not perform well. Thus, there is a need for absorbent articles with textured topsheets and open-cell foam absorbent layers that do not exhibit textural irregularities.

### SUMMARY

The present disclosure solves the problem of providing absorbent articles with textured topsheets and open-cell foam absorbent layers that do not exhibit textural irregularities.

Disclosed herein is an absorbent article including a liquid permeable topsheet, a liquid impervious backsheet, and an absorbent layer positioned therebetween. The topsheet includes a fibrous nonwoven material, an upper topsheet surface and a lower topsheet surface, and a first topsheet portion adjacent the upper topsheet surface and a second topsheet portion adjacent the lower topsheet surface. The absorbent layer includes a layer of open-cell foam having an upper foam surface that is in direct facing contact with the lower topsheet surface. The upper topsheet surface includes structured and unstructured regions. One or more unstructured regions of the upper topsheet surface exhibit a Sdcₐ of from 530 μm to 1000 μm when the topsheet is bonded to the absorbent layer with an adhesive. A portion of the adhesive penetrates into the second topsheet portion and the first topsheet portion is substantially free of adhesive.

Also disclosed is also directed to a package of two or more absorbent articles, the absorbent articles including a liquid permeable topsheet, a liquid impervious backsheet, and an absorbent layer positioned therebetween. The topsheet includes a fibrous nonwoven material, an upper topsheet surface and a lower topsheet surface, and a first portion adjacent the upper topsheet surface and a second portion adjacent the lower topsheet surface. The upper topsheet surface includes structured and unstructured regions. One or more regions of the upper topsheet surface exhibit a Sdcₐ of from 530 μm to 1000 μm when the topsheet is bonded to the absorbent layer with an adhesive. The absorbent layer includes a layer of open-cell foam having an upper foam surface that is in direct facing contact with the lower topsheet surface. A portion of the adhesive penetrates into the second topsheet portion and the first topsheet portion is substantially free of adhesive. The absorbent layer of at least one absorbent article is from 0.5 mm to 4 mm greater than an absorbent layer of another absorbent article in the package.

Also disclosed herein are methods of producing an absorbent article, the methods including obtaining a liquid permeable topsheet, a liquid impervious backsheet, and an absorbent layer. The topsheet includes a fibrous nonwoven material, an upper topsheet surface and a lower topsheet surface, and a first portion adjacent the upper topsheet surface and a second portion adjacent the lower topsheet surface. The upper topsheet surface includes structured and unstructured regions. One or more regions of the upper topsheet surface exhibit a Sdcₐ of from 530 μm to 1000 μm when the topsheet is bonded to the absorbent layer with an adhesive. The absorbent layer includes a layer of open-cell foam having an upper foam surface that is in direct facing contact with the lower topsheet surface. Methods further include applying adhesive to either the topsheet or the absorbent layer, and compressing the topsheet and the absorbent layer with a pressure. The pressure is selected so that the topsheet is at least partially attached to the absorbent layer using an adhesive, wherein the adhesive does not reach the first topsheet portion.

These and other features, aspects, and advantages will become better understood with reference to the following description and the appended claims.

Additional features and advantages of the configurations described herein will be set forth in the detailed description that follows, and in part will be readily apparent to those skilled in the art from that description or recognized by practicing the configurations described herein, including the detailed description that follows, the claims, as well as the appended drawings.

It is to be understood that both the foregoing general description and the following detailed description describe various configurations and are intended to provide an overview or framework for understanding the nature and character of the claimed subject matter. The accompanying drawings are included to provide a further understanding of the various configurations, and are incorporated into and constitute a part of this specification. The drawings illustrate the various configurations described herein, and together with the description serve to explain the principles and operations of the claimed subject matter.

### BRIEF DESCRIPTION OF THE DRAWINGS

FIG. 1 is a plan view of an example of a feminine hygiene pad, topsheet side facing the viewer.

FIG. 2 is a plan view of an example of an absorbent layer.

FIG. 3A is a schematic lateral cross section taken along lines 3-3 of the feminine hygiene pad of FIG. 1.

FIG. 3B is an enlarged portion 3B of the drawing of FIG. 3A, enlarged to depict the topsheet upper surface, the topsheet lower surface, the first topsheet portion, the second topsheet portion, the adhesive, the absorbent layer upper surface, the first portion of the absorbent layer, and the second portion of the absorbent layer.

FIGS. 4A-4C are plan views of several configurations of adhesive deposit patterns within a bonding region, in which a topsheet may be bonded to an absorbent layer.

FIG. 5 depicts the layout of the areas sampled in Example 1 and Example 2.

FIG. 6A is an SEM image of an example of an absorbent layer and topsheet wherein a portion of the adhesive penetrates into the first topsheet portion.

FIG. 6B is an SEM image of an example of an absorbent layer and topsheet wherein a portion of the adhesive penetrates into the second topsheet portion and the first topsheet portion is substantially free of adhesive according to configurations described herein.

FIG. 7 depicts the structured regions and unstructured regions of a topsheet according to configurations described herein.

### DETAILED DESCRIPTION

### Definitions

With respect to a feminine hygiene pad that is opened and laid out flat on a horizontal planar surface, “lateral” refers to a direction perpendicular to the longitudinal direction and parallel the horizontal planar surface.

With respect to a feminine hygiene pad that is opened and laid out flat on a horizontal planar surface and having a length measured from its forward end to its rearward end, “longitudinal” refers to a direction parallel with the line along which the length is measured, and parallel to the horizontal planar surface. “Length” refers to a dimension measured in the longitudinal direction.

With respect to a feminine hygiene pad, the terms “front,” “rear,” “forward” and “rearward” relate to features or regions of the pad corresponding to the position they would occupy when the pad is ordinarily worn by a user, and the front/anterior and rear/posterior of the user's body when standing.

With respect to a feminine hygiene pad that is opened and laid out flat on a horizontal planar surface, or a nonwoven web material (for example, a topsheet of nonwoven web material) laid out flat on a horizontal planar surface, “z-direction” refers to a direction perpendicular to the horizontal planar surface, and any plane parallel to the horizontal planar surface may be referred to as an “x-y plane”. When the pad is being worn by a user (and thus has been urged into a curving configuration), “z-direction” at any particular point location on the pad refers to a direction generally normal to the wearer-facing surface of the pad at the particular point location. With respect to a nonwoven web during its manufacture, “z-direction” refers to a direction orthogonal to both the machine direction and the cross direction of manufacture, and any plane parallel to the machine direction and cross direction may be referred to as an “x-y plane”.

With respect to a feminine hygiene pad, “wearer-facing” is a relative locational term referring to a feature of a component or structure of the pad that when in use that lies closer to the wearer than another feature of the component or structure that lies along the same z-direction. For example, a topsheet has a wearer-facing surface that lies closer to the wearer than the opposite, outward-facing surface of the topsheet.

With respect to a feminine hygiene pad, “outward-facing” is a relative locational term referring to a feature of a component or structure of the pad that when in use that lies farther from the wearer than another feature of the component or structure that lies along the same z-direction. For example, a topsheet has an outward-facing surface that lies farther from the wearer than the opposite, wearer-facing surface of the topsheet.

The relative location terms “inboard” and “outboard” refer to positioning of a first feature relative a second feature with respect to the lateral or longitudinal axis of a pad or layer component thereof, when both features are on the same side of the axis. For example, a first feature is laterally “inboard” of a second feature, and the second feature is laterally “outboard” of the first feature, when the first feature is closer to the longitudinal axis of the pad than the second feature. Similarly, a first feature is longitudinally “inboard” of a second feature, and the second feature is longitudinally “outboard” of the first feature, when the first feature is closer to the lateral axis of the pad than the second feature.

The terms “top,” “bottom,” “upper,” “lower,” “over,” “under,” “beneath,” “superadjacent,” “subjacent,” and similar terms relating to relative vertical positioning, when used herein to refer to layers, components or other features of an absorbent article such as a feminine hygiene pad, are relative the z-direction and are to be interpreted with respect to the pad as it would appear when laid out flat on a horizontal surface, with its wearer-facing surface oriented upward and outward-facing surface oriented downward.

### Description

Some segments of the consumer market for absorbent articles such as feminine hygiene pads prefer products with textured topsheets. However, when a textured topsheet is bonded to an absorbent layer that includes open-cell foam, the textured topsheet may become unevenly adhered to the absorbent layer. Without being bound by theory, it is believed that even minor variations in the absorbent layer thickness may contribute to this tendency for the textured topsheet to become unevenly adhered to the absorbent layer. While it is important to ensure that the topsheet is adhered to the absorbent layer, it has been discovered that compressing the topsheet and the absorbent layer too aggressively can cause inconsistent attachment of the topsheet to the absorbent layer. This issue can be compounded by variations in the thickness of the absorbent layer, as the variation in thickness can cause some portions of the absorbent layer to be compressed more than other layers. The inconsistent attachment of the topsheet to the absorbent layer may cause the topsheet to appear unappealing to the consumer, and may cause the consumer to assume that the absorbent article is of inferior quality, or that the absorbent article will not perform well.

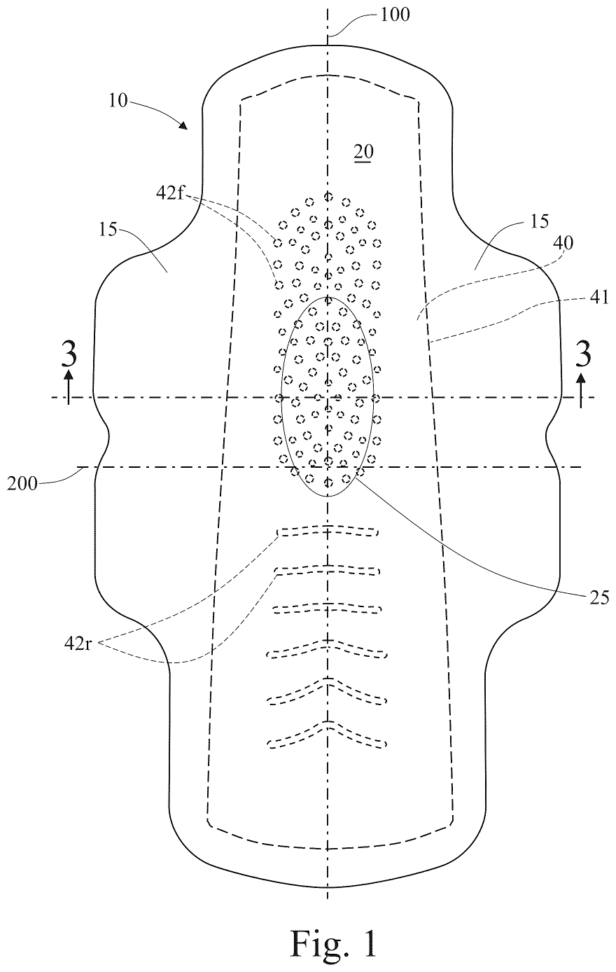

Referring to FIGS. 1, 2 and 3A, an absorbent article, such as a feminine hygiene pad 10 may include a liquid permeable topsheet 20, a liquid impervious backsheet 30 and an absorbent layer 40 disposed between the topsheet and the backsheet. The absorbent layer has an outer perimeter 41. In regions outside the outer perimeter 41, the topsheet and the backsheet may be bonded together in laminated fashion by any suitable mechanism including but not limited to adhesive bonding, thermal bonding, pressure bonding, etc., thereby enveloping, retaining and holding the absorbent layer 40 in place between the topsheet 20 and the backsheet 30. Absorbent layer 40 may be cut or otherwise imparted with a shape that is asymmetric about the lateral axis, as suggested in the figures, for purposes of enabling the cutting away of consecutive absorbent layers 40 from stock material along nested profiles that provide for efficient absorbent layer stock material utilization/minimization of cutoff scrap. It may be preferred that the laterally wider portion of the shape is in the rear of the pad, for purposes of providing more surface area to intercept discharged fluid that moves along a wearer's skin through the gluteal crevice. Feminine hygiene pad 10 may include opposing wing portions 15 extending laterally outside of perimeter 41 by a comparatively greater width dimension than the main portion of the pad. The outer surface of the backsheet forming the undersides of the main portion and the wing portions may have adhesive deposits 35 thereon. Adhesive deposits 35 may be provided to enable the user to adhere the pad to the inside of her underpants in the crotch region thereof, and wrap the wing portions through and around the inside edges of the leg openings of the underpants and adhere them to the outside/underside of the underpants in the crotch region, providing supplemental position holding support and helping guard the leg edges of the underpants against soiling. The inner surface of the backsheet forming the wing portions may have adhesive deposits thereon (not shown) and these adhesive deposits may adhere the backsheet to at least a portion of the topsheet or another layer in the wing portions. When feminine hygiene pad 10 is packaged, adhesive deposits 35 may be covered by one or more sheets of release film or paper (not shown) that covers/shields the adhesive deposits 35 from contact with other surfaces until the user is ready to remove the release film or paper and place the pad inside her underpants for use.

The absorbent article may have a basis weight of from about 100 grams per square meter (gsm) to about 600 gsm, from about 100 gsm to about 500 gsm, from about 100 gsm to about 400 gsm, from about 100 gsm to about 300 gsm, from about 100 gsm to about 200 gsm, from about 200 gsm to about 500 gsm, from about 200 gsm to about 400 gsm, from about 200 gsm to about 300 gsm, from about 300 gsm to about 600 gsm, from about 300 gsm to about 500 gsm, from about 300 gsm to about 400 gsm, from about 400 gsm to about 600 gsm, from about 400 gsm to about 500 gsm, or from about 500 gsm to about 600 gsm as measured according to the Absorbent Article Basis Weight Method described herein below.

The absorbent article may have a thickness of from about 0.5 mm to about 7 mm, from about 1 mm to about 7 mm, from about 2 mm to about 7 mm, from about 3 mm to about 7 mm, from about 4 mm to about 7 mm, from about 5 mm to about 7 mm, from about 6 mm to about 7 mm, from about 0.5 mm to about 6 mm, from about 1 mm to about 6 mm, from about 2 mm to about 6 mm, from about 3 mm to about 6 mm, from about 4 mm to about 6 mm, from about 5 mm to about 6 mm, from about 0.5 mm to about 5 mm, from about 1 mm to about 5 mm, from about 2 mm to about 5 mm, from about 3 mm to about 5 mm, from about 4 mm to about 5 mm, from about 0.5 mm to about 4 mm, from about 1 mm to about 4 mm, from about 2 mm to about 4 mm, from about 3 mm to about 4 mm, from about 0.5 mm to about 3 mm, from about 1 mm to about 3 mm, from about 2 mm to about 3 mm, from about 0.5 mm to about 2 mm, from about 1 mm to about 2 mm, or from about 0.5 mm to about 1 mm measured according to the Thickness Test described herein below.

The thickness of the absorbent article may be variable in portions of the absorbent article. The thickness of the absorbent article may have a standard deviation within the absorbent article of from about 0.01 mm to about 0.5 mm, from about 0.01 mm to about 0.4 mm, from about 0.01 mm to about 0.3 mm, from about 0.01 mm to about 0.25 mm, from about 0.01 mm to about 0.2 mm, from about 0.01 mm to about 0.1 mm, from about 0.01 mm to about 0.05 mm, from about 0.05 mm to about 0.5 mm, from about 0.05 mm to about 0.4 mm, from about 0.05 mm to about 0.3 mm, from about 0.05 mm to about 0.25 mm, from about 0.05 mm to about 0.2 mm, from about 0.05 mm to about 0.1 mm, from about 0.1 mm to about 0.5 mm, from about 0.1 mm to about 0.4 mm, from about 0.1 mm to about 0.3 mm, from about 0.1 mm to about 0.25 mm, from about 0.1 mm to about 0.2 mm, from about 0.2 mm to about 0.5 mm, from about 0.2 mm to about 0.4 mm, from about 0.2 mm to about 0.3 mm, from about 0.2 mm to about 0.25 mm, from about 0.25 mm to about 0.5 mm, from about 0.25 mm to about 0.4 mm, from about 0.25 mm to about 0.3 mm, from about 0.3 mm to about 0.5 mm, from about 0.3 mm to about 0.4 mm, or from about 0.4 mm to about 0.5 mm.

### Topsheet

Topsheet 20 may be formed of a suitable nonwoven web material that is compliant, soft feeling, and non-irritating to wearers' skin. Referring back to the figures, the topsheet 20 is positioned adjacent a wearer-facing surface of the absorbent layer 40 and may be joined thereto and to the backsheet 30 by any suitable attachment or bonding method. The topsheet 20 and the backsheet 30 may be joined directly to each other in the peripheral regions outside the outer perimeter 41 of the absorbent layer 40 and may be indirectly joined by directly joining them respectively to wearer-facing and outward-facing surfaces of the absorbent layer or additional optional layers included with the pad.

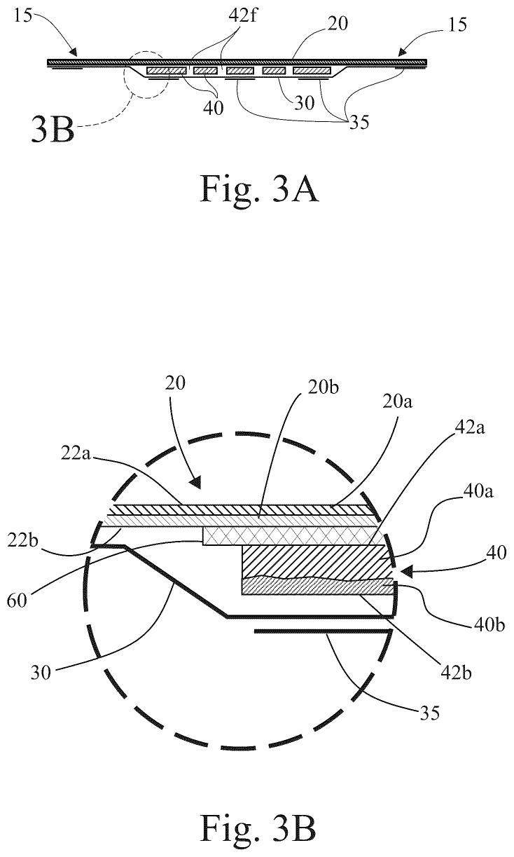

Referring now to FIG. 3B, topsheet 20 may be liquid permeable. Topsheet 20 may include a fibrous nonwoven material, an upper topsheet surface 22a and a lower topsheet surface 22b, and a first topsheet portion 20a adjacent the upper topsheet surface 22a and a second topsheet portion 20b adjacent the lower topsheet surface 22b. The first topsheet portion 20a may be wearer-facing and the second topsheet portion 20b may be outward-facing.

A suitable topsheet material will include a liquid pervious material that is comfortable when in contact with the wearer's skin and under suitable circumstances will permit discharged menstrual fluid to rapidly move through it. A suitable topsheet may be made of various materials such as nonwoven web materials.

As contemplated herein, component fibrous nonwoven material from which topsheet 20 may be cut may be a fibrous nonwoven material that includes or consists predominately (by weight) of fibers spun from polymeric resin such as polyolefins and/or polyesters, including but not limited polypropylene, polyethylene, polyethylene terephthalate (PET) and variants, blends, and bicomponent or multicomponent arrangements thereof. Topsheet component nonwoven web material also may include in partial or predominant weight fraction natural fibers such as but not limited to cotton, hemp, kapok, bamboo, etc.

The nonwoven web may be formed via any suitable process by which spun fibers of indefinite and/or staple lengths may be distributed and accumulated in a controlled fashion onto a moving forming belt to form a batt having a desired distribution of the fibers, to a desired basis weight. Suitable processes may include spunbonding and meltblowing (for fibers of indefinite, relatively greater length), and carding or airlaying (for fibers of staple, relatively shorter length) or co-forming (for a mix of fibers of indefinite lengths and fibers of staple lengths). After accumulation, the batt may be processed to consolidate and bond the fibers into a cohesive web by any suitable method, including calendering, calender thermal bonding, calender compression bonding, through-air bonding, etc. The batt or consolidated web may also be subjected to processes such as hydroenhancing or hydroentangling, to impart web cohesiveness via inter-fiber entanglement, increase z-direction orientation of fibers, and/or increase loft.

Absent enhancements to the materials and/or processes involved, generally, monocomponent fibers spun from polymer resin tend to have relatively simple surface geometry, typically an approximately round or oval-shaped cross section, and a substantially non-curled or non-crimped configuration along the lengths thereof. As a consequence, when the spun fibers are deposited and accumulated on a forming belt, calendered and bonded (e.g., in a spunbonding process), the resulting nonwoven web product will have relatively low loft and a relatively macroscopically flat and untextured appearance, as compared with a web of a comparable basis weight formed of more complexly-shaped, e.g., curled or crimped, fibers. Some consumers may perceive a relatively lower loft nonwoven web to have a relatively less pleasing feel and appearance, i.e., it may be perceived to be, relatively, not as soft or luxurious, as a higher-loft one.

To add loft to the web without increasing basis weight (and material usage), and to increase opacity of the web, the fibers used to make the web may be spun in multicomponent, e.g., bicomponent, fiber configurations. The fibers may be natural or synthetic. Resin-processing equipment and beams of spinnerets may be configured, and polymer resins may be selected, to spin bicomponent fibers that crimp or curl as they leave the spinnerets as molten polymer streams, and subsequently cool and solidify into fibers. Known processes and polymer resin selections may be used to produce curly spun bicomponent fibers wherein the fibers have side-by-side, eccentric core-sheath, or other non-coaxial polymer component cross-sectional configurations. In such non-coaxial configurations, one of the polymer components may be selected and/or formulated to have a differing melting temperature and/or cooling contraction rate than the other polymer component. Upon cooling, the differing properties of the polymer components and non-coaxial cross-sectional arrangement of component sections of the molten fiber streams impart curl to the fibers as they cool, contract at differing rates and solidify. The respective polymer resin components may be differing polymers, differing forms or variants of the same polymers, or differing blends thereof. More detailed disclosure of spinning curled or crimped bicomponent fibers and forming a nonwoven web thereof may be found in, for example, U.S. Pat. No. 8,501,646; EP 1 988 793; and US 2007/0275622. In some configurations bicomponent fibers may have respective predominately polypropylene-based resin components formulated to impart differing melting temperatures to the respective components. In some configurations bicomponent fibers may have respective components in which one component is predominately polypropylene-based, and the other component is predominately polyethylene-based. In some more particular configurations the bicomponent fibers may be spun with an eccentric core-sheath component configuration wherein a predominately polypropylene-based component is the core component and a predominately polyethylene-based component is the sheath component; wherein the polypropylene-based component may be desired for its greater tensile strength, and the polyethylene-cased component may be desired for its smoother, more lubricious surface feel, that helps impart a silky feel to the fiber and to the nonwoven web material. It will be appreciated that other combinations of polyolefins and/or other spinnable thermoplastic resins may be selected for their differing cooling contraction rates and other differing qualities that affect the qualities (including curl or crimp) and properties of the spun fibers in differing ways.

Another potential advantage of inclusion of bicomponent fibers is that, with appropriate selection of the component resins for the fibers, the fibers in the web may be bonded to each other at random locations via application of heat or heated air-through bonding, wherein the fibers are heated to an extent sufficient to cause them to partially melt and fuse together at locations where they contact each other. The fiber component resins may be selected such that a first component resin has a lower melting temperature than the second component resin. The web may be heated to an extent sufficient to cause the first component resin but not the second component resin to partially melt, such that the first components of contacting fibers will fuse, without causing the second components to lose their shape. In this manner, the web may be imparted with added resiliency and mechanical (tensile) strength, without a loss of z-direction thickness/loft that would be otherwise caused by z-direction compression as occurs in other bonding processes such as, for example, calender bonding. In some configurations, bicomponent fibers having a sheath-core configuration may be provided with a sheath component having a relatively lower melting temperature and a core component having a relatively higher melting temperature. Using a heat treatment as described above, the web may be bonded such that the sheaths of the fibers fuse, without melting of the cores. In some configurations, the sheath components may be formed of or include a polyethylene having a lower melting temperature, and the core components may be formed of or include a polypropylene or a polyethylene terephthalate (PET) having a higher melting temperature.

The topsheet may further incorporate or include any features of topsheets described in U.S. patent application Ser. Nos. 16/789,516 and/or 16/789,522.

Many commercially practical thermoplastic resins that may be desired to process and spin into bicomponent fibers are normally hydrophobic. Such resins may include polyolefins such as polypropylene and polyethylene. A nonwoven web material formed of such fibers will also be hydrophobic, and as such, will not readily accept or wick aqueous fluid such as menstrual fluid. When such resins are used, therefore, additional measures can be included to render the fibers and/or the nonwoven web, or portions thereof, hydrophilic. In some configurations, a suitable surfactant may be applied to the nonwoven web following its formation. In more particular configurations, a suitable surfactant finish used may be SILASTOL PHP 26, a product of Schill+Seilacher GmbH, Böblingen, Germany. Other suitable surfactants may include STANTEX spin finishes (Pulcra Chemicals, Geretsried, Germany), for example, STANTEX 6887; other SILASTOL spin finishes, for example PHP26, PHP28, PHP90, PHP207, PST-N, etc. In addition or alternatively, resin-incorporated surfactants (i.e., melt additives) may be added to polymer resin prior to fiber spinning, which can impart and/or increase hydrophilicity. Suitable melt additives may include PPM15560, PM19668 and PPM112172 (Techmer PM, Clinton, Tennessee), or VW351 or S-1416 (Polyvel Inc., Hammonton, New Jersey). Staple fibers in carded webs may have spin finishes provided as supplied to impart the desired surface energy as well as other characteristics.

In some configurations, it may be desired to selectively render or treat fibers of portions of the topsheet nonwoven web material with agents or additives to impart a combination of enhanced hydrophilicity and enhanced hydrophobicity, according to particular spatial, i.e., z-direction and/or x-y planar locations or regions of the topsheet. For example, it may be desired that fibers proximate the wearer-facing top side of the web be predominately hydrophobic, and that fibers proximate the underside of the topsheet be predominately hydrophilic. In such configurations, this may be desired to cause underlying fibers to attract and draw fluid from overlying fibers, and wick the fluid down to the underlying absorbent layer, while causing overlying fibers to be more resistive to rewetting by movement of fluid back up to the wearer-facing surface. In such configurations, the fibers of the web may be inherently hydrophobic and/or imparted with enhanced hydrophobicity by inclusion of hydrophobicity-increasing melt additives in the component polymer resin, prior to spinning. Suitable melt additives may include, for example erucamide or glycerol tristearate. Following manufacture, the underside of the topsheet material may be selectively treated, across its entire area, or a sub-portion thereof, with a hydrophilicity-enhancing agent. Examples are described in US App. Pub. No. 2019/0388578.

The finish may be applied to the web using any suitable method, for example, via kiss roll coater. The finish may be applied in a quantity suitable to impart the nonwoven web with a desired level of hydrophilicity and thereby help impart it with a desired wicking capability. In particular configurations, a finish coating of SILASTOL PHP 26 may be applied in a quantity sufficient to constitute, after drying, surfactant weight quantity that is 0.30 percent to 0.60 percent, more preferably 0.40 percent to 0.50 percent of the basis weight of the nonwoven web material.

Wicking performance also may vary according to, and may be manipulated by, the manner in which the web is further processed. Factors such as level of consolidation (i.e., densification) of the fiber mass in the end structure and orientations of the individual fibers within the end structure can affect absorbency and wicking performance.

Thus, for purposes contemplated herein, in combination with being imparted with a suitable basis weight, density and/or thickness as discussed above, it may be desired that nonwoven web material formed in part or in substantial entirety of fibers spun from thermoplastic polymer resin and used to make the topsheet, be formed via a nonwoven web manufacturing process in which substantial portions of the fibers are imparted with directional orientation that includes some z-direction orientation, rather than orientations predominately biased along the machine direction or x-y plane of formation of the web structure. Following any suitable processes in which fibers are distributed and laid down in a batt on a horizontal forming belt (e.g., via a spunbond process), additional process steps that forcibly reorient some of the fibers or portions thereof in the z-direction may be employed. Suitable process steps may include needle punching and hydroentangling or hydroenhancing. Hydroentangling or hydroenhancing, in which an array of fine, high-velocity water jets is directed at the batt as it is conveyed past them, may be desired for its effectiveness in reorienting lengths of fibers while breaking fewer fibers and creating less broken fiber lint and surface fuzz (free fiber ends extending from the surface of the web). The batt is conveyed past the water jets on a belt or drum having a surface constituted by a permeable fine mesh or screen having a pattern of orifices or pores, through which the jetted water may be drawn. The belt or drum surface may also include a pattern or other configuration of protrusions and/or recesses arranged to cause the web to be imparted with textural features in the water-jetting step. A vacuum water removal system may be operably disposed on the back side or underside of the belt or drum surface, to draw the jetted water therethrough, and can tend to create, add, open and/or clear small z-direction passageways within the fiber matrix of the web, approximately in the pattern of the orifices or pores. Without intending to be bound by theory, it is believed that the portions of the fibers oriented in the z-direction and the z-direction passageways increase the ability and tendency of the web to wick aqueous fluid in the z-direction. In a topsheet, this would mean that the material can more readily wick aqueous fluid from the wearer-facing surface of the topsheet to the outward-facing surface of the topsheet, i.e., down to the absorbent layer below, and may thereby wick fluid less along x-y planar directions (causing a stain from discharged fluid to spread laterally and/or longitudinally).

### Nonwoven Topsheet Texture Characteristics

It is believed that consumers/users of absorbent articles of the types contemplated herein (including feminine hygiene pads) appreciate visible 3D texture in the visible wearer-facing surface of the product. However, it is believed that, to date, a thin and pliable feminine hygiene pad that has a nonwoven web topsheet directly overlying a layer of absorbent foam to serve functions of receiving and absorbing discharged menstrual fluid, has not been offered with a topsheet having appreciable, visible 3D texture. It is believed that, prior to the research and experimentation described herein, it was generally believed in the art that a textured nonwoven topsheet (myriad examples have been manufactured and are available for purchase) would be unsuitable for combination with directly subjacent foam absorbent layer, because it would compromise fluid acquisition performance.

It has been learned, however, that a nonwoven web material exhibiting a desired 3D patterned texture pattern on one side thereof, while being relatively less-textured or even substantially less-textured, non-textured or flat on the other side, may be manufactured. Through prototyping and experimentation it has been learned that such a material can be successfully combined with an open-cell foam absorbent layer, wherein the textured side is disposed to the wearer-facing side of the article, and the relatively less-textured side is disposed to face the foam absorbent layer. In connection with this effort, techniques have been discovered that enable the characterization and identification of suitable nonwoven web material, and distinguishing a textured nonwoven web material that is suitable, from others that are unsuitable. By “suitable” in this context, it is meant that the nonwoven web material has 3D texture or topographic features on one side that are readily visible to the naked eye, while having a relatively less-textured side that does not unacceptably compromise the ability of the combination of topsheet and foam to receive and absorb discharged menstrual fluid at an acceptable rate. The topsheet may include a pattern. The pattern may be formed from an arrangement of structured and unstructured zones.

In a series of patent applications that include those having publication numbers: US2017/0027774; US2017/0029993; US2017/0191198; WO2017/105997; US2017/0029994; US2018/0168893; WO2018/112144; WO2018/112146; US2018/0216269; US2018/0216271; US2018/0216270; US2018/0214318; US2020/0268572; US2019/0003079; US2019/0003080; US2019/0374405; US2019/0374407; US2019/0374388; US2019/0380887; US2019/0298587; US2019/0298586; US2020/0299880; US2020/0299881; US2020/0345563; US2020/0347533; US2020/0397629; US2020/0397630; US2020/0100956; US2021/0169710; US2021/0369511; US2021/0369512; and US2022/0192897, and PCT/CN2023/073146, methods for manufacturing nonwoven web materials with formed, ordered patterns of alternating built-up regions and attenuated regions are disclosed. These methods do not include or require embossing of the web material. As explained and depicted in more detail in, for example, US2019/0380887, U.S. Pat. Nos. 10,765,565 and 11,547,613, the built-up regions are regions of the nonwoven material in which constituent spun fiber count and numerical density per unit x-y planar surface area are relatively greater; and the attenuated regions are regions in which constituent spun fiber count and numerical density per unit x-y planar surface area are relatively lesser. The regions of built-up fiber count and numerical density also have visibly relatively greater z-direction thickness, while the attenuated regions have visibly relatively lesser z-direction thickness. The forming belt or drum used in the method may be adapted so as to impart a desired pattern to the built-up regions and attenuated regions, and thus, desired visible textural features.

Generally, this nonwoven structure may be manufactured by entraining continuous spun polymer filaments (i.e., continuous fibers—herein the terms “filaments” and “fibers” are used interchangeably) into a continuous air stream. The air stream and thus the entrained filaments are directed to a working location on a nonwoven web manufacturing line, at which a continuously cycling forming belt or rotating forming drum are located. The receiving surface of the cycling forming belt or rotating forming drum cycles and travels through the working location, along a machine direction. The receiving surface of the forming belt or drum has an ordered pattern of airflow-permeable regions and airflow-blocked regions formed thereon. The air-flow permeable regions are formed by a screen-type structure with apertures or pores that are numerous enough to allow air to pass through the receiving surface relatively freely, and small enough to substantially prevent entrained filaments from being carried through with the air. A vacuum system is disposed opposite the receiving surface and draws the air in the air stream through the airflow-permeable regions. As a result, as the entrained filaments are carried toward the receiving surface, and they are drawn to, strike, and are accumulated on, the receiving surface, they accumulate more heavily over the air-permeable regions, and less heavily over the airflow-blocked regions on the receiving surface. The resulting batt having built-up regions and attenuated regions of accumulated filaments is then separated from the receiving surface for further processing downstream. The areas of heavier accumulation (built-up regions) and areas of lighter accumulation (attenuated regions) correspond to the ordered pattern of airflow-permeable regions and airflow-blocked regions formed on the receiving surface of the belt or drum. It has been learned that, when the receiving surface of the forming belt or drum is suitably configured such that the airflow-permeable regions are located within pockets or surface depressions and the airflow-blocked regions include raised areas or protrusions, the resulting formed nonwoven batt will be “sided,” having a substantial macroscopic 3D texture on the side that was furthest from the air/entrained filament source (consisting more of filaments that were accumulated earlier in time, more heavily in the airflow permeable regions of the receiving surface), and substantially reduced macroscopic 3D texture (greater flatness) on the side that was closest to the air/entrained filament source (consisting more of fibers that were accumulated later in time, to overlie the earlier-accumulated filaments). Calendering of the batt and calender bonding, if desired, in downstream processes, can help to further flatten the less-textured/flatter side. For purposes herein, a nonwoven material manufactured in this manner will be referred to as a “Patterned Fiber Accumulation” (PFA) material.

It has been learned through experimentation that a topsheet made from a nonwoven web material manufactured in this manner may be combined with an absorbent foam layer, to form an absorbent article such as a feminine hygiene pad, with a wearer-facing topsheet surface having desirable macroscopic 3D texture or topographical features, wherein the ability of the pad to receive and absorb menstrual fluid at a sufficient rate is still achieved. Since fluid moves through a topsheet nonwoven material by flowing along surfaces of constituent fibers, it is desirable to ensure that as many fiber surfaces as possible are in contact amongst themselves and directly or indirectly with fiber surfaces contacting with the upper surface of the subjacent foam layer, so that the fluid will rapidly wick along fiber surfaces down through the topsheet, and then contact the foam layer and be drawn into it. A relatively flatter lower topsheet surface facing a subjacent foam absorbent layer will provide for relatively greater contact area between the fibers defining the lower topsheet surface, and the foam layer.

In connection with this effort and discovery, methods for characterizing desired 3D texture features of a nonwoven web material, which forms a topsheet of an absorbent article, have been identified. Given the myriad 3D texture designs that might be imagined and implemented through configuration of nonwoven web forming equipment, the methods enable the identification of a material that will be successful in presenting a visible, macroscopic 3D texture to the user, while exhibiting parity with or improvement on the fluid handling characteristics (fluid receiving and absorption performance, and low rewetting) of currently-marketed, foam-based pads that lack substantial 3D topsheet texture. These methods are set forth below, and are used to measure “Sdc” and “Sdr %”. “Sdcₜ” and “Sdrₜ %” refer to Sdc and Sdr % of the topsheet when it is not adhered to the absorbent layer or the Sdc and Sdr % of the absorbent core when it is not adhered to the topsheet. “Sdcₐ” and “Sdrₐ %” refer to Sdc and Sdr % of the topsheet when it is adhered to the absorbent layer.

Generally, the measured value of Sdc reflects an average magnitude of a difference in z-direction height between highest points and lowest points of the topography of one side of a nonwoven web material. Thus, a measured Sdc of a higher magnitude reflects a more dramatically textured topography in terms of height differences between highest “peaks” and lowest “valleys” of the topography.

Generally, the measured value of Sdr % reflects the difference between macroscopic x-y planar surface area as viewed along a z-direction, and surface area of all of the topographic features, along all surface contours they define. It is expressed as a percent ratio of surface area along surface contours, to macroscopic x-y planar surface area. To illustrate the concept, a mountain on Earth has a horizontal/projected two-dimensional x-y planar surface area as viewed directly along a vertical (z) direction from space, but also contour surface area in three dimensions (3D), theoretically measurable by physically walking along, measuring and tallying the area along all contours that its land surface defines. The steeper and/or more complex the surface topography of the mountain, the greater is its ratio of its contour surface area to its horizontal/projected x-y planar surface area; conversely, the flatter and/or more simple the surface topography of the mountain, the lesser is its ratio of its contour surface area to its horizontal/projected x-y planar surface. Thus, for a topsheet, the ratio quantifies the extent to which topsheet 3D topography/contour surface area differs from that of a horizontal/projected plane of the same x-y dimensions. For a topsheet nonwoven, the measured and calculated ratio that follows this analysis is Sdr %.

Because the “surface”, or side, of a nonwoven is actually made up, at a microscopic level, of the many portions of surfaces of the fibers that are outermost in the z-direction proximate that side, it will be appreciated that these measurement methods and the values measured will depend to some extent upon the resolution size selected for the imaging equipment used in the measurement method. If the selected resolution is too high, the measurements will reflect differences in positions among individual fibers throughout the entire z-direction thickness of the nonwoven, rather than more macroscopic surface contours/texture (or flatness) of one side of the material. Conversely, if the selected resolution is too low, the measurements will not make sufficient distinction between a highly textured surface and a relatively flat one, to enable a determination whether a particular nonwoven material is suitable for purposes contemplated herein. Accordingly, for purposes herein, a resolution size has been selected for use in the measurement methods, which is deemed appropriate for characterizing and measuring topsheet topography to generate data deemed suitable to reflect visibility of enhanced topsheet topography to the naked eye, and to reflect flatness of topsheet topography for purposes effectuating rapid transfer of fluid from the topsheet to an underlying absorbent foam layer. To ensure consistency of Sdcₐ measurement across different patterns, Sdcₐ measurements are conducted on unstructured regions of the topsheet or foam core when the topsheet or foam core included both structured and unstructured regions, as explained in further detail hereinbelow.

From prototyping and experimentation with various topsheet materials including PFA material produced by the process described above as well as examples of spunbond and carded/spunlaced/hydrojetted materials imparted with surface texture via hydrojetting over drums with patterned forming surfaces, it has been determined that a combination of topsheet nonwoven with an absorbent foam layer directly therebeneath (i.e., subjacent), will be attractive to consumers who prefer topsheet texture, while performing at parity or better as compared with current market products with respect to fluid acquisition and absorption rates, when the foam layer wearing-facing (upper) surface, the topsheet outward-facing (lower) surface, and the topsheet wearer-facing (upper) surface are manufactured such that their measured values for Sdcₜ and Sdrₜ % conform to the following limits.

A foam layer exhibiting values conforming to the limits for Sdcₜ and Sdrₜ % shown in Table 1, above, may be manufactured as described below.

TABLE 1

| | Sdcₜ (μm) | Sdrₜ % |
|---|---|---|
| Topsheet | >360 more | >1.0 more |
| upper surface | preferably >420 | preferably >1.1 |
| Topsheet | <350 more | <0.80 more |
| lower surface | preferably <300 | preferably <0.750 |
| Foam layer | <200 more | <0.100 more |
| upper surface | preferably <150 | preferably <0.050 |

Alternatively, or in combination, the values reflecting the magnitude of texture/contouring of the topsheet lower surface may be kept within a maximum difference exceeding that of the foam layer upper surface, as follows.

In Table 2, “ΔSdcₜ” is the difference between the measured value of Sdcₜ for the lower surface of the topsheet material, and the measured value of Sdcₜ for the upper surface of the absorbent foam layer. “ΔSdcₜ %” is the ratio of ΔSdcₜ to the measured value of Sdcₜ for the upper surface of the absorbent foam layer, times 100%.

TABLE 2

| | ΔSdcₜ (μm) | ΔSdcₜ % |
|---|---|---|
| Topsheet | <250 more | <250 more |
| lower surface | preferably <200 | preferably <200 |

The upper topsheet surface may exhibit an Sdcₜ at least 15% greater or at least 20% greater than the lower topsheet surface Sdcₜ. The upper topsheet surface may exhibit an Sdcₜ 1000% less than the lower topsheet surface Sdcₜ.

In Table 3, “ΔSdrₜ %” is the difference between the measured value of Sdrₜ % for the lower surface of the topsheet material, and the measured value of Sdrₜ % for the upper surface of the absorbent foam layer. “ΔSdrₜ % %” is the ratio of ΔSdrₜ % to the measured value of Sdrₜ % for the upper surface of the absorbent foam layer, times 100%.

TABLE 3

| | ΔSdrₜ % | ΔSdrₜ % % |
|---|---|---|
| Topsheet | <1.00 more | <5,000 more |
| lower surface | preferably <0.800 | preferably <3,500 |

One or more unstructured regions of the upper topsheet surface may exhibit a Sdcₐ of from about 530 μm to about 1000 μm, such as from about 540 μm to about 1000 μm, from about 550 μm to about 1000 μm, from about 560 μm to about 1000 μm, from about 570 μm to about 1000 μm, from about 580 μm to about 1000 μm, from about 590 μm to about 1000 μm, from about 600 μm to about 1000 μm, from about 610 μm to about 1000 μm, from about 620 μm to about 1000 μm, from about 630 μm to about 1000 μm, from about 640 μm to about 1000 μm, from about 650 μm to about 1000 μm, from about 660 μm to about 1000 μm, from about 670 μm to about 1000 μm, from about 530 μm to about 950 μm, from about 530 μm to about 900 μm, from about 530 μm to about 850 μm, from about 530 μm to about 800 μm, or from about 530 μm to about 750 μm, when the topsheet is bonded to the absorbent layer with an adhesive.

The topsheet 20 may be bonded to the absorbent layer 40 using an adhesive 60. Referring to FIG. 3B, a portion of the adhesive 60 may penetrate into the second topsheet portion 20b and the first topsheet portion 20a may be substantially free of, or free of adhesive. For ease of viewing, the second topsheet portion 20b and the adhesive 60 are shown as separate layers. It is to be understood that the topsheet 20 may be a single layer material or may comprise two or more sublayers bonded together. The first topsheet portion 20a may comprises less than 10 weight percent (wt. %), less than 5 wt. %, less than 2 wt. %, less than 1 wt. %, less than 0.5 wt. %, or less than 0.1 wt. % adhesive. It has been surprisingly found that when adhesive 60 used to adhere the topsheet to the absorbent layer bleeds through the first topsheet portion 20a and on to the upper topsheet surface 22a, the topsheet may exhibit textural irregularities that are caused by the topsheet 20 being unevenly bonded to the absorbent layer 40. These textural irregularities may be especially problematic with some patterned topsheets, as the appearance of the pattern may appear to enhance or emphasize the presence of the textural irregularities. Furthermore, the textural irregularities may cause the pattern to appear inconsistent. Consumers may interpret these textural irregularities to mean that the absorbent article is of inferior quality, or that the absorbent article will not perform well.

Furthermore, the textural irregularities caused by adhesive bleeding through the topsheet upper surface may cause the topsheet upper surface Sdcₐ in one or more unstructured regions to be lower than the topsheet upper surface would be if the adhesive did not bleed through the first topsheet portion and on to the upper topsheet surface. Without being bound by theory, it is believed that having adhesive in the first topsheet portion causes the first topsheet portion, and in some cases, the upper topsheet surface, to become bonded directly to the absorbent layer. However, when the first topsheet portion is substantially free of adhesive, the first topsheet portion and the upper topsheet surface are not bonded directly to the absorbent layer and the first topsheet portion and upper topsheet surface are able to provide loft to the wearer-facing surface of the topsheet. However, it is critical that the at least some of the adhesive adheres to the topsheet lower surface, otherwise the topsheet may not be sufficiently bonded to the absorbent layer, which could lead to the consumer to perceive that the absorbent article is defective. In some cases, it may also be beneficial to the adhesion of the topsheet to the absorbent layer if the adhesive penetrates into the second topsheet portion. If the topsheet is not sufficiently bonded to the absorbent layer, the topsheet can detach from the absorbent layer during use, which can cause increased leaks and/or larger stains because fluid cannot effectively reach the absorbent layer.

At least a portion of the adhesive 60 may penetrate, by depth, from about 1% to about 70%, from about 5% to about 70%, from about 10% to about 70%, from about 20% to about 70%, from about 25% to about 70%, from about 30% to about 70%, from about 40% to about 70%, from about 50% to about 70%, from about 60% to about 70%, from about 1% to about 60%, from about 5% to about 60%, from about 10% to about 60%, from about 20% to about 60%, from about 25% to about 60%, from about 30% to about 60%, from about 40% to about 60%, from about 50% to about 60%, from about 1% to about 50%, from about 5% to about 50%, from about 10% to about 50%, from about 20% to about 50%, from about 25% to about 50%, from about 30% to about 50%, from about 40% to about 50%, from about 1% to about 40%, from about 5% to about 40%, from about 10% to about 40%, from about 20% to about 40%, from about 25% to about 40%, from about 30% to about 40%, from about 1% to about 30%, from about 5% to about 30%, from about 10% to about 30%, from about 20% to about 30%, from about 25% to about 30%, from about 1% to about 25%, from about 5% to about 25%, from about 10% to about 25%, from about 20% to about 25%, from about 1% to about 20%, from about 5% to about 20%, from about 10% to about 20%, from about 1% to about 10%, from about 5% to about 10%, or from about 1% to about 5% into the topsheet.

The first topsheet portion may include from about 1% to about 30%, from about 5% to about 30%, from about 10% to about 30%, from about 20% to about 30%, from about 25% to about 30%, from about 1% to about 25%, from about 5% to about 25%, from about 10% to about 25%, from about 20% to about 25%, from about 1% to about 20%, from about 5% to about 20%, from about 10% to about 20%, from about 1% to about 10%, from about 5% to about 10%, or from about 1% to about 5% of the topsheet depth.

The second topsheet portion may include from about 1% to about 70%, from about 5% to about 70%, from about 10% to about 70%, from about 20% to about 70%, from about 25% to about 70%, from about 30% to about 70%, from about 40% to about 70%, from about 50% to about 70%, from about 60% to about 70%, from about 1% to about 60%, from about 5% to about 60%, from about 10% to about 60%, from about 20% to about 60%, from about 25% to about 60%, from about 30% to about 60%, from about 40% to about 60%, from about 50% to about 60%, from about 1% to about 50%, from about 5% to about 50%, from about 10% to about 50%, from about 20% to about 50%, from about 25% to about 50%, from about 30% to about 50%, from about 40% to about 50%, from about 1% to about 40%, from about 5% to about 40%, from about 10% to about 40%, from about 20% to about 40%, from about 25% to about 40%, from about 30% to about 40%, from about 1% to about 30%, from about 5% to about 30%, from about 10% to about 30%, from about 20% to about 30%, from about 25% to about 30%, from about 1% to about 25%, from about 5% to about 25%, from about 10% to about 25%, from about 20% to about 25%, from about 1% to about 20%, from about 5% to about 20%, from about 10% to about 20%, from about 1% to about 10%, from about 5% to about 10%, or from about 1% to about 5% of the topsheet depth.

At its thickest point, the topsheet may have a thickness at 7 g/cm² pressure of from about 0.1 mm to about 5 mm, from about 0.2 mm to about 5 mm, from about 0.5 mm to about 5 mm, from about 1 mm to about 5 mm, from about 2 mm to about 5 mm, from about 0.1 mm to about 4 mm, from about 0.2 mm to about 4 mm, from about 0.5 mm to about 4 mm, from about 1 mm to about 4 mm, from about 2 mm to about 4 mm, from about 0.1 mm to about 3 mm, from about 0.2 mm to about 3 mm, from about 0.5 mm to about 3 mm, from about 1 mm to about 3 mm, from about 2 mm to about 3 mm, from about 0.1 mm to about 2 mm, from about 0.2 mm to about 2 mm, from about 0.5 mm to about 2 mm, from about 1 mm to about 2 mm, from about 0.1 mm to about 1 mm, from about 0.2 mm to about 1 mm, from about 0.5 mm to about 1 mm, from about 0.1 mm to about 0.5 mm, from about 0.2 mm to about 0.5 mm, or from about 0.1 mm to about 0.2 mm, as measured according to the Thickness-Pressure Method.

The topsheet may be an apertured topsheet or an unapertured topsheet. Both apertured and unapertured topsheets may include bond points, attenuated regions, or combinations thereof. The bond points may be ultrasonic bonds, pressure bonds, thermal bonds, or combinations thereof. The apertures may be any shape or size, including linear, circular, square, diamond shaped, heart shaped, oval, star shaped, rectangular, or any other shape. The bond points may be any shape or size, including linear, circular, square, diamond shaped, heart shaped, oval, star shaped, rectangular, or any other shape. The apertures, bond points, or combinations thereof may be arranged to form structured regions and unstructured regions.

As used herein, a “structured region” refers to regions that include bond points, attenuated regions, apertures, or combinations thereof. As used herein, an “unstructured region” refers to regions that are substantially free of bond points, attenuated regions, apertures, or combinations thereof. The bond points, attenuated regions, apertures, or combinations thereof are sufficiently small and/or close together so that a viewer sees distinct, visually identifiable, structured and unstructured regions. An unstructured region may be discrete, with one or more structured regions forming a perimeter around the unstructured zone. Likewise, a structured region may be discrete, with one or more unstructured regions forming a perimeter around the structured zone. The structured regions and unstructured regions may be arranged to form patterns.

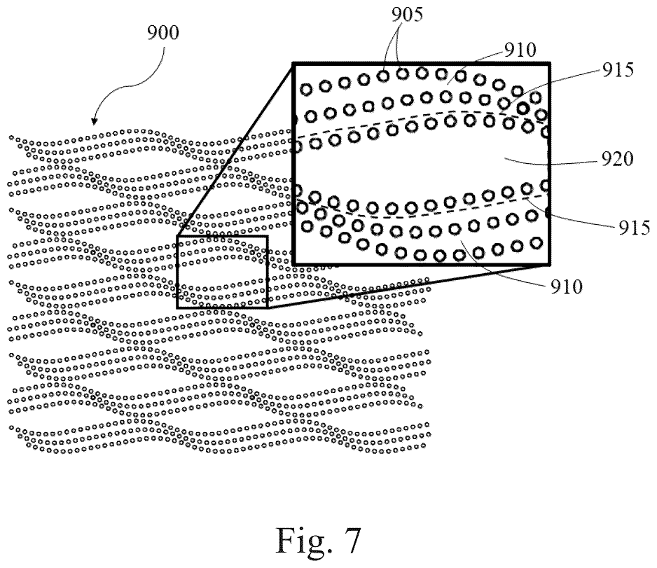

Referring now to FIG. 7, a portion of a topsheet with a textured pattern is shown. The pattern 900 includes bond points 905. The bond points form structured regions 910 and unstructured regions 920.

Textural irregularities may form in the unstructured regions. The extent of the textural irregularities may be determined by measuring the Sdcₐ of one or more unstructured regions. The Sdcₐ of the one or more unstructured regions may be determined by selecting a surface area to be analyzed that includes an unstructured region with a sufficient number of bond points, attenuated regions, apertures, or combinations thereof in an adjacent structured region to form a border. The total area of the individual bond points, attenuated regions, apertures, or combinations thereof equates to approximately 5% of the selected area. For example, referring again to FIG. 7, the unstructured region 920 selected for imaging should include the adjacent portions of structured regions 910. Thus, the selected unstructured region is defined by boundary line 915.

### Absorbent Layer

The absorbent layer 40 may have a thickness of from about 1.5 mm to about 10 mm, from about 2 mm to about 10 mm, from about 3 mm to about 10 mm, from about 4 mm to about 10 mm, from about 5 mm to about 10 mm, from about 6 mm to about 10 mm, from about 7 mm to about 10 mm, from about 8 mm to about 10 mm, from about 9 mm to about 10 mm, from about 1.5 mm to about 9 mm, from about 2 mm to about 9 mm, from about 3 mm to about 9 mm, from about 4 mm to about 9 mm, from about 5 mm to about 9 mm, from about 6 mm to about 9 mm, from about 7 mm to about 9 mm, from about 8 mm to about 9 mm, from about 1.5 mm to about 8 mm, from about 2 mm to about 8 mm, from about 3 mm to about 8 mm, from about 4 mm to about 8 mm, from about 5 mm to about 8 mm, from about 6 mm to about 8 mm, from about 7 mm to about 8 mm, from about 1.5 mm to about 7 mm, from about 2 mm to about 7 mm, from about 3 mm to about 7 mm, from about 4 mm to about 7 mm, from about 5 mm to about 7 mm, from about 6 mm to about 7 mm, from about 1.5 mm to about 6 mm, from about 2 mm to about 6 mm, from about 3 mm to about 6 mm, from about 4 mm to about 6 mm, from about 5 mm to about 6 mm, from about 1.5 mm to about 5 mm, from about 2 mm to about 5 mm, from about 3 mm to about 5 mm, from about 4 mm to about 5 mm, from about 1.5 mm to about 4 mm, from about 2 mm to about 4 mm, from about 3 mm to about 4 mm, from about 1.5 mm to about 3 mm, from about 2 mm to about 3 mm, or from about 1.5 mm to about 2 mm as determined according to the Thickness Test described herein below.

The thickness of the absorbent layer 40 may be variable throughout portions of absorbent layer 40. The absorbent layer 40 may have a standard deviation of thickness within the absorbent layer 40 of about 0.01 mm to about 0.5 mm, from about 0.01 mm to about 0.4 mm, from about 0.01 mm to about 0.3 mm, from about 0.01 mm to about 0.25 mm, from about 0.01 mm to about 0.2 mm, from about 0.01 mm to about 0.1 mm, from about 0.01 mm to about 0.05 mm, from about 0.05 mm to about 0.5 mm, from about 0.05 mm to about 0.4 mm, from about 0.05 mm to about 0.3 mm, from about 0.05 mm to about 0.25 mm, from about 0.05 mm to about 0.2 mm, from about 0.05 mm to about 0.1 mm, from about 0.1 mm to about 0.5 mm, from about 0.1 mm to about 0.4 mm, from about 0.1 mm to about 0.3 mm, from about 0.1 mm to about 0.25 mm, from about 0.1 mm to about 0.2 mm, from about 0.2 mm to about 0.5 mm, from about 0.2 mm to about 0.4 mm, from about 0.2 mm to about 0.3 mm, from about 0.2 mm to about 0.25 mm, from about 0.25 mm to about 0.5 mm, from about 0.25 mm to about 0.4 mm, from about 0.25 mm to about 0.3 mm, from about 0.3 mm to about 0.5 mm, from about 0.3 mm to about 0.4 mm, or from about 0.4 mm to about 0.5 mm.

Within a feminine hygiene pad 10, the absorbent layer 40 may have a first thickness in one portion and a second thickness in another portion, wherein the first thickness and the second thickness vary by from about 0 mm to about 4 mm, from about 0.5 mm to about 4 mm, from about 1 mm to 4 about mm, from about 1.5 mm to about 4 mm, from about 2 mm to about 4 mm, from about 2.5 mm to about 4 mm, from about 3 mm to about 4 mm, from about 0 mm to about 3 mm, from about 0.5 mm to about 3 mm, from about 1 mm to about 3 mm, from about 1.5 mm to about 3 mm, from about 2 mm to about 3 mm, from about 2.5 mm to about 3 mm, from about 0 mm to about 2.5 mm, from about 0.5 mm to about 2.5 mm, from about 1 mm to about 2.5 mm, from about 1.5 mm to about 2.5 mm, from about 0 mm to about 2 mm, from about 0.5 mm to about 2 mm, from about 1 mm to about 2 mm, from about 0 mm to about 1.5 mm, from about 0.5 mm to about 1.5 mm, from about 0 mm to about 1 mm, or from about 0.5 mm to about 1 mm as determined according to the Thickness Test described hereinbelow.

Referring to FIG. 3B, the absorbent layer 40 may include a first absorbent portion 40a with an upper foam surface 42a and a second absorbent portion 40b with a lower foam surface 42b. In some configurations, the adhesive may not reach the second absorbent portion. The adhesive may penetrate by depth, from about 5% to about 100%, from about 10% to about 100%, from about 20% to about 100%, from about 25% to about 100%, from about 30% to about 100%, from about 40% to about 100%, from about 50% to about 100%, from about 60% to about 100%, from about 70% to about 100%, from about 75% to about 100%, from about 80% to about 100%, from about 90% to about 100%, from about 95% to about 100%, from about 5% to about 99%, from about 10% to about 99%, from about 20% to about 99%, from about 25% to about 99%, from about 30% to about 99%, from about 40% to about 99%, from about 50% to about 99%, from about 60% to about 99%, from about 70% to about 99%, from about 75% to about 99%, from about 80% to about 99%, from about 90% to about 99%, from about 95% to about 99%, from about 5% to about 95%, from about 10% to about 95%, from about 20% to about 95%, from about 25% to about 95%, from about 30% to about 95%, from about 40% to about 95%, from about 50% to about 95%, from about 60% to about 95%, from about 70% to about 95%, from about 75% to about 95%, from about 80% to about 95%, from about 90% to about 95%, from about 5% to about 90%, from about 10% to about 90%, from about 20% to about 90%, from about 25% to about 90%, from about 30% to about 90%, from about 40% to about 90%, from about 50% to about 90%, from about 60% to about 90%, from about 70% to about 90%, from about 75% to about 90%, about from 80% to about 90%, from about 5% to about 80%, from about 10% to about 80%, from about 20% to about 80%, from about 25% to about 80%, from about 30% to about 80%, from about 40% to about 80%, from about 50% to about 80%, from about 60% to about 80%, from about 70% to about 80%, from about 75% to about 80%, from about 5% to about 75%, from about 10% to about 75%, from about 20% to about 75%, from about 25% to about 75%, from about 30% to about 75%, from about 40% to about 75%, from about 50% to about 75%, from about 60% to about 75%, from about 70% to about 75%, from about 5% to about 70%, from about 10% to about 70%, from about 20% to about 70%, from about 25% to about 70%, from about 30% to about 70%, from about 40% to about 70%, from about 50% to about 70%, from about 60% to about 70%, from about 5% to about 60%, from about 10% to about 60%, from about 20% to about 60%, from about 25% to about 60%, from about 30% to about 60%, from about 40% to about 60%, from about 50% to about 60%, from about 5% to about 50%, from about 10% to 50%, from about 20% to about 50%, from about 25% to about 50%, from about 30% to about 50%, from about 40% to about 50%, from about 5% to about 40%, from about 10% to about 40%, from about 20% to about 40%, from about 25% to about 40%, from about 30% to about 40%, from about 5% to about 30%, from about 10% to about 30%, from about 20% to about 30%, from about 25% to about 30%, from about 5% to about 25%, from about 10% to about 25%, from about 20% to about 25%, from about 5% to about 20%, from about 10% to about 20%, or from about 5% to about 10% into the absorbent layer.

Without being bound by theory, it is believed that when the adhesive penetrates a greater percentage into the absorbent layer, it may lead to increased adhesive bond strength between the topsheet and the absorbent layer, as a greater force may be needed to detach the topsheet from the absorbent layer.

In some configurations, the absorbent layer 40 may be formed of or include a layer of absorbent open-celled foam material. The absorbent layer 40 may include a layer of open-cell foam having an upper foam surface 42a that is in direct facing contact with the lower topsheet surface 22b. In some configurations, the foam material may comprise one or more sublayers being in direct face-to-face contact with each other. In some configurations, a wearer-facing sublayer may be a relatively larger-celled foam material, and an outward-facing sublayer may be a relatively smaller-celled foam material.

The open-celled foam material may be a foam material that is manufactured via polymerization of the continuous oil phase of a water-in-oil high internal phase emulsion (“HIPE”). Exemplary water-in-oil high internal phase emulsions may include those described in US2024/0115436 and US2024/0156647A1, all of which are incorporated by reference herein. Exemplary HIPE foams can be found in ALWAYS brand and Liquid Pad (which may be referred to as “Ye Ti— [symbol] ” in Chinese) brand pads manufactured by the Procter and Gamble Company.

A water-in-oil HIPE has two phases. One phase is a continuous oil phase comprising monomers to be polymerized, and an emulsifier to help stabilize the HIPE. The monomer component may be included in an amount of from about 80% to about 99% by weight of the oil phase. The emulsifier component, which is soluble in the oil phase and suitable for forming a stable water-in-oil emulsion may be included in the oil phase in an amount of from about 1% to about 20% by weight of the oil phase.

In general, the monomers may include substantially water-insoluble monofunctional alkyl acrylate or alkyl methacrylate. For example, monomers of this type may include C4-C18 alkyl acrylates and C2-C18 methacrylates, such as ethylhexyl acrylate, butyl acrylate, hexyl acrylate, octyl acrylate, nonyl acrylate, decyl acrylate, isodecyl acrylate, tetradecyl acrylate, benzyl acrylate, nonyl phenyl acrylate, hexyl methacrylate, 2-ethylhexyl methacrylate, octyl methacrylate, nonyl methacrylate, decyl methacrylate, isodecyl methacrylate, dodecyl methacrylate, tetradecyl methacrylate, and octadecyl methacrylate.

The oil phase may also include from about 2% to about 40% by weight of the oil phase, a substantially water-insoluble, polyfunctional crosslinking comonomer. Examples of crosslinking monomers of this type can include monomers containing two or more activated acrylate, methacrylate groups, or combinations thereof. Examples of this group can include 1,6-hexanedioldiacrylate, 1,4-butanedioldimethacrylate, trimethylolpropane triacrylate, trimethylolpropane trimethacrylate, 1,12-dodecyldimethacrylate, 1,14-tetradecanedioldimethacrylate, ethylene glycol dimethacrylate, neopentyl glycol diacrylate (2,2-dimethylpropanediol diacrylate), hexanediol acrylate methacrylate, glucose pentaacrylate, sorbitan pentaacrylate, and the like.

Any third substantially water-insoluble comonomer may be added to the oil phase to modify properties of the HIPE foams. In certain cases, “toughening” monomers may be desired to impart toughness to the resulting HIPE foam. These can include monomers such as styrene, vinyl chloride, vinylidene chloride, isoprene, and chloroprene. Monomers may also be added to confer flame retardancy, as disclosed, for example, in U.S. Pat. No. 6,160,028. Monomers may be added to impart color (for example vinyl ferrocene); to impart fluorescent properties; to impart radiation resistance; to impart opacity to radiation (for example lead tetraacrylate); to disperse charge; to reflect incident infrared light; to absorb radio waves; to make surfaces of the HIPE foam struts or cell walls wettable; or for any other desired property in a HIPE foam.

The oil phase may further include an emulsifier to stabilize the HIPE. Emulsifiers used in a HIPE can include: (a) sorbitan monoesters of branched C16-C24 fatty acids; linear unsaturated C16-C22 fatty acids; and linear saturated C12-C14 fatty acids, such as sorbitan monooleate, sorbitan monomyristate, and sorbitan monoesters, sorbitan monolaurate diglycerol monooleate, polyglycerol monoisostearate, and polyglycerol monomyristate; (b) polyglycerol monoesters of -branched C16-C24 fatty acids, linear unsaturated C16-C22 fatty acids, or linear saturated C12-C14 fatty acids, such as diglycerol monooleate (for example diglycerol monoesters of C18:1 fatty acids), diglycerol monomyristate, diglycerol monoisostearate, and diglycerol monoesters; (c) diglycerol monoaliphatic ethers of -branched C16-C24 alcohols, linear unsaturated C16-C22 alcohols, and linear saturated C12-C14 alcohols, and mixtures of these emulsifiers. See U.S. Pat. Nos. 5,287,207 and 5,500,451. Another emulsifier that may be used is polyglycerol succinate, which is formed from an alkyl succinate, glycerol, and triglycerol.

Such emulsifiers, and combinations thereof, may be added to the oil phase so that they constitute about 1% to about 20% of the weight of the oil phase. In certain configurations, coemulsifiers may also be used to provide additional control of cell size, cell size distribution, and emulsion stability. Examples of coemulsifiers may include phosphatidyl cholines and phosphatidyl choline-containing compositions, aliphatic betaines, long chain C12-C22 dialiphatic quaternary ammonium salts, short chain C1-C4 dialiphatic quaternary ammonium salts, long chain C12-C22 dialkoyl(alkenoyl)-2-hydroxyethyl, short chain C1-C4 dialiphatic quaternary ammonium salts, long chain C12-C22 dialiphatic imidazolinium quaternary ammonium salts, short chain C1-C4 dialiphatic imidazolinium quaternary ammonium salts, long chain C12-C22 monoaliphatic benzyl quaternary ammonium salts, long chain C12-C22 dialkoyl(alkenoyl)-2-aminoethyl, short chain C1-C4 monoaliphatic benzyl quaternary ammonium salts, short chain C1-C4 monohydroxyaliphatic quaternary ammonium salts, ditallow dimethyl ammonium methyl sulfate, and combinations thereof.

The oil phase and/or the aqueous phase may comprise a photoinitiator. Exemplary photoinitiators may include those described in U.S. Patent Publication No. 2024/0156647A1.

The dispersed aqueous phase of a HIPE comprises water, and may also comprise one or more components, such as initiator, photoinitiator, or electrolyte, wherein in certain configurations, the one or more components are at least partially water soluble.

One component included in the aqueous phase may be a water-soluble electrolyte. The water phase may contain from about 0.2% to about 40% by weight of the aqueous phase of a water-soluble electrolyte. The electrolyte minimizes the tendency of monomers, comonomers, and crosslinkers that are primarily oil soluble to also dissolve in the aqueous phase. Examples of electrolytes may include chlorides or sulfates of alkaline earth metals such as calcium or magnesium and chlorides or sulfates of alkali earth metals such as sodium. Such electrolyte can include a buffering agent for the control of pH during the polymerization, including such inorganic counterions as phosphate, borate, and carbonate, and mixtures thereof.

Another component that may be included in the aqueous phase is a water-soluble free-radical initiator. The initiator can be present at up to about 20 mole percent based on the total moles of polymerizable monomers present in the oil phase. Examples of suitable initiators may include ammonium persulfate, sodium persulfate, potassium persulfate, 2,2′-azobis(N,N′-dimethyleneisobutyramidine)dihydrochloride, azo initiators, redox couples like persulfate-bisulfate, persulfate-ascorbic acid, other suitable redox initiators, and combinations thereof.

HIPE foam is produced from the polymerization of the monomers comprising the continuous oil phase of a HIPE. In certain configurations, a HIPE foam layer may have one or more sublayers, and may be either homogeneous or heterogeneous polymeric open-celled foams. Homogeneity and heterogeneity relate to distinct layers within the same HIPE foam, which are similar in the case of homogeneous HIPE foams and differ in the case of heterogeneous HIPE foams. A heterogeneous HIPE foam may contain at least two distinct sublayers that differ with regard to their chemical composition, physical properties, or both; for example, sublayers may differ with regard to one or more of foam density, polymer composition, specific surface area, or pore size (also referred to as cell size). For example, for a HIPE foam if the difference relates to pore size, the average pore size in the respective sublayers may differ by at least about 20%, in certain configurations by at least about 35%, and in still other configurations by at least about 50%. In another example, if the differences in the sublayers of a HIPE foam layer relate to density, the densities of the layers may differ by at least about 20%, in certain configurations by at least about 35%, and in still other configurations by at least about 50%. For instance, if one layer of a HIPE foam has a density of 0.020 g/cc, another layer may have a density of at least about 0.024 g/cc or less than about 0.016 g/cc, in certain configurations at least about 0.027 g/cc or less than about 0.013 g/cc, and in still other configurations at least about 0.030 g/cc or less than about 0.010 g/cc. If the differences between the layers are related to the chemical composition of the HIPE or HIPE foam, the differences may reflect a relative amount difference in at least one monomer component, for example by at least about 20%, in certain configurations by at least about 35%, and in still further configurations by at least about 50%. For instance, if one sublayer of a HIPE or HIPE foam is composed of about 10% styrene in its formulation, another sublayer of the HIPE or HIPE foam may be composed of at least about 12%, and in certain configurations of at least about 15%.

A HIPE foam layer structured to have distinct sublayers formed from differing HIPEs may provide a HIPE foam layer with a range of desired performance characteristics. For example, a HIPE foam layer comprising first and second foam sublayers, wherein the first foam sublayer has a relatively larger pore or cell size, than the second sublayer, when used in an absorbent article may more quickly absorb incoming fluids than the second sublayer. For example, when the HIPE foam layer is used to form an absorbent layer of a feminine hygiene pad, the first foam sublayer may be layered over the second foam sublayer having relatively smaller pore sizes, as compared to the first foam sublayer, which exert more capillary pressure and draw the acquired fluid from the first foam sublayer, restoring the first foam sublayer's ability to acquire more fluid from above. HIPE foam pore sizes may range from 1 to 200 μm and in certain configurations may be less than 100 μm. HIPE foam layers of the present disclosure having two major parallel surfaces may be from about 0.5 to about 10 mm thick, and in certain configurations from about 2 to about 10 mm. The desired thickness of a HIPE foam layer will depend on the materials used to form the HIPE foam layer, the speed at which a HIPE is deposited on a belt, and the intended use of the resulting HIPE foam layer.

The HIPE foam layers of the present disclosure are relatively open-celled. This refers to the individual cells or pores of the HIPE foam layer being in substantially unobstructed communication with adjoining cells. The cells in such substantially open-celled HIPE foam structures have intercellular openings or windows that are large enough to permit ready fluid transfer from one cell to another within the HIPE foam structure. For purpose of the present disclosure, a HIPE foam is considered “open-celled” if at least about 80% of the cells in the HIPE foam that are at least 1 μm in size are in fluid communication with at least one adjoining cell.

In certain configurations, for example when it is used to form an absorbent layer of a feminine hygiene pad, a HIPE foam layer may be flexible and exhibit an appropriate glass transition temperature (Tg). The Tg represents the midpoint of the transition between the glassy and rubbery states of the polymer. In general, HIPE foams that have a Tg that is higher than the temperature of use can be strong but will also be relatively rigid and potentially prone to fracture (brittle). In certain configurations, regions of the HIPE foams of the current disclosure which exhibit either a relatively high Tg or excessive brittleness will be discontinuous. Since these discontinuous regions will also generally exhibit high strength, they can be prepared at lower densities without compromising the overall strength of the HIPE foam.

HIPE foams intended for applications requiring flexibility should contain at least one continuous region having a Tg as low as possible, so long as the overall HIPE foam has acceptable strength at in-use temperatures. In certain configurations, the Tg of this region will be less than about 40° C. for foams used at about ambient temperature conditions; in certain other configurations Tg will be less than about 30° C. For HIPE foams used in applications wherein the use temperature is higher or lower than ambient temperature, the Tg of the continuous region may be no more than 10° C. greater than the use temperature, in certain configurations the same as use temperature, and in further configurations about 10° C. less than use temperature wherein flexibility is desired. Accordingly, monomers are selected as much as possible that provide corresponding polymers having lower Tg's.

HIPE foams useful for forming absorbent layers and/or sublayers within contemplation of the present disclosure, and methods for their manufacture, also include but are not necessarily limited to those foams and methods described in U.S. Pat. Nos. 10,752,710; 10,045,890; 9,056,412; 8,629,192; 8,257,787; 7,393,878; 6,551,295; 6,525,106; 6,550,960; 6,406,648; 6,376,565; 6,372,953; 6,369,121; 6,365,642; 6,207,724; 6,204,298; 6,158,144; 6,107,538; 6,107,356; 6,083,211; 6,013,589; 5,899,893; 5,873,869; 5,863,958; 5,849,805; 5,827,909; 5,827,253; 5,817,704; 5,817,081; 5,795,921; 5,741,581; 5,652,194; 5,650,222; 5,632,737; 5,563,179; 5,550,167; 5,500,451; 5,387,207; 5,352,711; 5,397,316; 5,331,015; 5,292,777; 5,268,224; 5,260,345; 5,250,576; 5,149,720; 5,147,345; and US 2005/0197414; US 2005/0197415; US 2011/0160326; US 2011/0159135; US 2011/0159206; US 2011/0160321; and US 2011/0160689, which are incorporated herein by reference to the extent not inconsistent herewith.

It will be appreciated that, when the absorbent foam layer material is cured or polymerized from a liquidous emulsion that has been deposited over a smooth, flat and level forming surface as described in references cited above, e.g., U.S. Pat. No. 10,752,710, because the emulsion is liquidous and of sufficiently low viscosity to enable it to substantially seek its own level over the forming surface, the resulting foam will have smooth and flat top and bottom surfaces following curing/polymerization, reflecting the smooth and flat forming surface on the bottom and the smooth and flat, self-leveled top surface of the precursor liquidous emulsion. The top/upper surface will typically exhibit Sdcₜ and Sdrₜ % values easily conforming to the ranges set forth in Table 1, above.

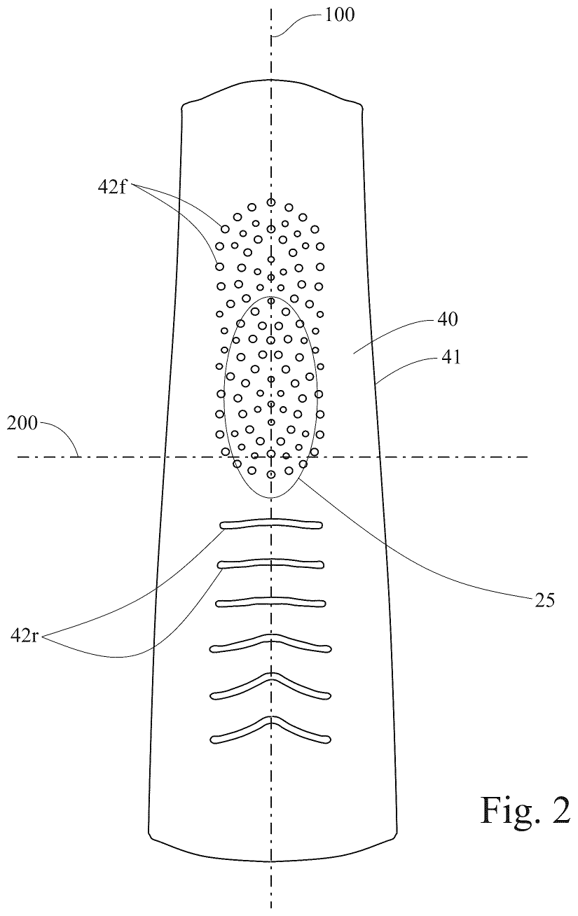

As reflected in FIGS. 1 and 2, the absorbent layer 40 formed of HIPE foam may include one or more patterns of forward and rearward perforations 42f, 42r, including at least a first pattern disposed within an expected discharge location proximate the intersection of longitudinal and lateral axes 100, 200 of the pad. Perforations 42f, 42r may be punched, cut, molded, or otherwise formed through the entire z-direction depth of the HIPE foam absorbent layer, or only through a wearer-facing layer or partially into the wearer-facing portion thereof. When a HIPE foam absorbent layer is disposed in direct contact with a topsheet as described herein, with no intervening acquisition layer formed of another material, perforations 42f, 42r may serve as a group of “reservoirs” to receive, temporarily hold, and aid in distributing rapid discharges of relatively small quantities of menstrual fluid, until the HIPE foam has sufficient time to distribute and absorb the fluid via capillary action. Additionally, such perforations help decrease bending stiffness of the absorbent layer, which may help increase comfort of the pad for the wearer. A pattern of perforations having an average radius or other largest dimension of about 1.0 mm to about 4.0 mm, or from about 1.5 mm to about 3.5 mm may be included, within, for example, the area occupied by a bonding region 25. The pattern may include perforations at a numerical density of about 3.0 to about 9.0 perforations per cm², or about 4.0 to about 8.0 perforations per cm². In selecting the appropriate average size, numerical density, and surface area occupied by the pattern of perforations, the manufacturer may wish to balance the volume of the “reservoirs” desired with the need to retain absorbent material in locations proximate to and about the expected discharge location. Additional details concerning configurations of such perforations in combination with configurations of suitable absorbent layers may be found in U.S. Pat. No. 8,211,078.

Preferably, the topsheet 20 is bonded to the wearer-facing surface of the absorbent layer 40 in a manner that assures close proximity between the two, to provide for rapid fluid movement down through the topsheet to the absorbent layer 40, while not creating an unacceptable degree of occlusion (created, e.g., by overly large deposits of adhesive between these component) that would obstruct downward fluid movement. Bonding the topsheet to the absorbent layer also helps fix the absorbent layer 40, and helps unitize the overall structure of the pad, enhancing user/wearer impressions of quality. The topsheet 20 may be bonded to the absorbent layer 40 in any suitable manner, including that described in U.S. patent application Ser. No. 16/789,522.

### Bonding Between Topsheet and Absorbent Layer

In configurations in which the topsheet material includes component fibers of sufficient hydrophilicity to cause it to wick, it may tend to retain fluid on its surfaces, and within the interstitial spaces between and along the surfaces of the fibers of the web material, unless there is sufficient direct contact maintained between the topsheet and the underlying absorbent layer to enable the fluid to move from fiber surfaces within the topsheet structure, directly to surfaces of material of the underlying absorbent layer. Prior to the time it is fully saturated, a nonwoven web material may not release retained fluid unless an adjacent material (with greater affinity for the fluid) is in sufficient direct contact. Accordingly, it is important to provide structure sufficient to maintain sufficient contact between the topsheet and the underlying absorbent layer, without obstructing fluid movement. No intervening layer or structure of material, or at least no intervening layer or structure of material less absorbent that that of the absorbent layer, should be interposed between the material of the topsheet 20 and the material of the absorbent layer 40, at least within the bonding region 25, more preferably over a majority of the wearer-facing surface area of the absorbent layer 40, and even more preferably over the entirety of the wearer-facing surface area of the absorbent layer 40—unlike systems provided in many current feminine hygiene pads, which include a distinct fluid acquisition/distribution material layer between the topsheet and the absorbent materials of the absorbent layer.

In some configurations, sufficient direct contact between the topsheet 20 and the absorbent layer 40 may be effected by deposit(s) of adhesive between the topsheet and the absorbent layer, adhesively bonding them in close z-direction proximity. The adhesive may be applied in a pattern or arrangement of adhesive deposits interspersed with areas in which no adhesive is present (unbonded areas), such that the adhesive holds the two layers in close z-direction proximity, while areas remain in which no adhesive is present to obstruct z-direction fluid movement between the layers.

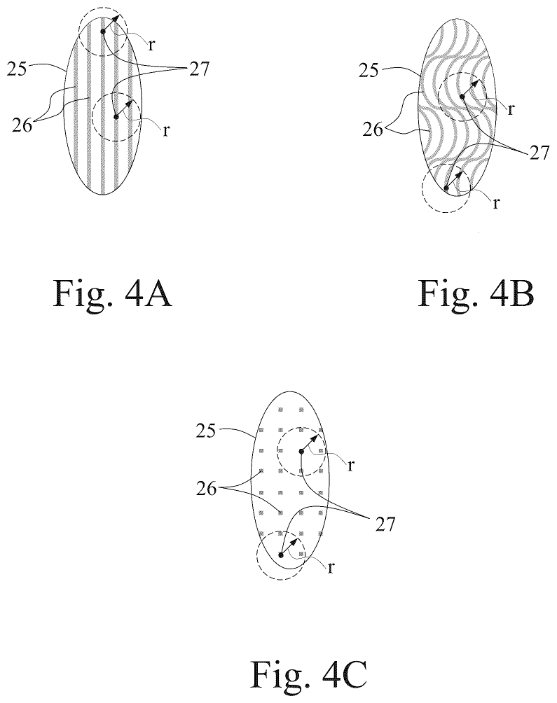

Referring to FIGS. 1 and 4A-4C, to ensure that the topsheet and absorbent layer are held in sufficiently close z-direction proximity at least in the area of the topsheet expected to receive a discharge of fluid, it may be desired to dispose a bonding region 25 on the pad at a location that includes the intersection of the longitudinal and lateral axes 100, 200. The bonding region 25 should be of sufficient size to be reliably present beneath the expected discharge location when the pad is in use, with reasonably minor variations of placement by the user/wearer within the underpants; accordingly, it may be desired that the bonding region have an area of at least 15 cm², or at least 30 cm². In some configurations, it may be desired that the bonding region have an area that is at least half of the total wearer-facing surface area of the absorbent layer. (Note: FIGS. 4A-4C are not presented herein as actual size or scale depictions.)

To ensure that the topsheet 20 and the absorbent layer 40 remain in sufficient z-direction proximity during use, it may be desired that, within any identifiable first point location 27 within the bonding region, at which the topsheet is bonded to the absorbent layer, there is a second point location at which the topsheet is bonded to the absorbent layer, within a 10 mm radius, more preferably within a 6 mm radius, 5 mm radius, 4 mm radius, and even more preferably within a 3 mm radius r of the first point location. Referring to FIGS. 4A-4C, depicting three non-limiting configurations, it can be seen that a variety of patterns or arrangements of bonds (via adhesive deposits 26 or other bonding mechanisms) may be employed to impart this feature. Within radius r of each point location 27, there are a number of additional point locations where bonding between the topsheet and the absorbent layer is present in the configurations depicted.

It will be appreciated that a continuous deposit of adhesive may be applied to bond the topsheet and the absorbent layer 40 within the entirety of bonded region 25, but that such a continuous deposit of adhesive could form a barrier that would obstruct the movement of fluid from the topsheet to the absorbent layer. Accordingly, it is preferable that, in configurations in which the bonding mechanism is deposits of adhesive, the deposits are disposed in a pattern or arrangement that is discontinuous or intermittent such that it creates bonded areas interspersed with unbonded areas between the topsheet and the absorbent layer. Additionally, when the absorbent layer is formed of an open-celled foam (such as a HIPE foam contemplated herein) it may be desired that the adhesive selected not effect adhesion to the absorbent layer via chemical, dispersive or diffusive adhesion with the foam layer at the adhesive deposit locations, but rather, that it effect adhesion to the foam layer mechanically, by flowing to a limited extent into the cells, at least partially assuming the shapes thereof, and solidifying in such position to form mechanical interlocks with the cell structures, which enable the adhesive to hold the topsheet to the absorbent layer. Such an adhesive may be preferred so as not to alter the molecular structure or composition of the foam material, potentially negatively affecting its fluid absorption properties or mechanical strength. The adhesive may be a hot melt adhesive. In one example, a suitable adhesive for use with a HIPE foam may be H1750 hot melt adhesive from Bostik, Wauwatosa, Wisconsin (currently a subsidiary of Arkema, Columbes, France). The hot melt adhesive may be present in the absorbent article at a basis weight of from about 2 grams per square meter (gsm) to about 9 gsm, from about 4 gsm to about 9 gsm, from about 6 gsm to about 9 gsm, from about 2 gsm to about 7 gsm, from about 4 gsm to about 7 gsm, from about 5 gsm to about 6 gsm, or from about 2 gsm to about 4 gsm.

Suitable adhesives may include fiberized adhesives, spray adhesives, or combinations thereof. In examples where a fiberized adhesive is used, the fiberized adhesive may have an average fiber thickness of from about 1 μm to about 100 μm and an average fiber length of from about 5 mm to about 50 cm.

The adhesive may be applied uniformly so that adhesive is distributed evenly throughout the absorbent article. The adhesive may be applied irregularly so that specific area or zones of the absorbent article lack adhesive while other areas or zones include adhesive. Different areas or zones may include different amounts of adhesive, or may lack adhesive entirely.

The adhesive may provide an adhesive bond strength of from about 0.1 N/25 mm to about 1.0 N/25 mm, from 0.2 N/25 mm to 1.0 N/25 mm, from 0.4 N/25 mm to 1.0 N/25 mm, from 0.5 N/25 mm to 1.0 N/25 mm, from 0.6 N/25 mm to 1.0 N/25 mm, from 0.8 N/25 mm to 1.0 N/25 mm, from 0.1 N/25 mm to 0.8 N/25 mm, from 0.2 N/25 mm to 0.8 N/25 mm, from 0.4 N/25 mm to 0.8 N/25 mm, from 0.5 N/25 mm to 0.8 N/25 mm, from 0.6 N/25 mm to 0.8 N/25 mm, from 0.1 N/25 mm to 0.6 N/25 mm, from 0.2 N/25 mm to 0.6 N/25 mm, from 0.4 N/25 mm to 0.6 N/25 mm, from 0.5 N/25 mm to 0.6 N/25 mm, from 0.1 N/25 mm to 0.5 N/25 mm, from 0.2 N/25 mm to 0.5 N/25 mm, from 0.4 N/25 mm to 0.5 N/25 mm, from 0.1 N/25 mm to 0.4 N/25 mm, from 0.2 N/25 mm to 0.4 N/25 mm, or from 0.1 N/25 mm to 0.2 N/25 mm as measured by the Standard Test Method for Peel Resistance of Adhesives (T-Peel Test) ASTM D-1876.

A central bonding region straddling the intersection of longitudinal and lateral axes, the central bonding region having a size of at least 15 cm², within which the topsheet is bonded to the absorbent layer via a discontinuous pattern of adhesive bonds, and within which, within any identifiable first point location of an adhesive bond within the bonding region, at which the topsheet is bonded to the absorbent layer, there is a second point location at which the topsheet is bonded to the absorbent layer by adhesive, within a 10 mm radius r of the first point location.

Unapertured topsheets for feminine hygiene pads formed of nonwoven web material and including or consisting predominately of hydrophilic fibers are known and have been included with some feminine hygiene products to date. (Herein, an “unapertured” nonwoven topsheet is one in which a majority of its surface area has not been subjected to any process that creates an arrangement of holes or apertures entirely therethrough, that persist prior to wetting of the topsheet, of an average size (greatest dimension) greater than about 0.5 mm along an x-y planar direction.) Although favored by some consumers for their pleasant feel against the skin, topsheets formed of hydrophilic nonwoven web material have been disfavored by other consumers as a result of their substantial absorbency, i.e., capillary absorption and desorption pressures, causing them to resist drainage by conventionally included acquisition/distribution and absorbent layer structures. Following a discharge of menstrual fluid, a pad with such a topsheet overlying a conventional absorbent structure can feel to the user like a wet cloth held against the skin for an extended time period, which many users find objectionable. This dilemma has been present in the field for many years.

It has been discovered, however, that an unapertured fibrous nonwoven topsheet overlaid in direct, sufficient face-to-face proximate relationship with a HIPE foam absorbent layer or other layer adapted/manufactured to have capillary absorption capability sufficient to draw fluid from the topsheet, without any intervening less absorbent layers and in combination with other structural features as described herein, will be substantially drained of fluid by the absorbent layer, and regain a much drier feel against the skin following a discharge. It has been discovered that a suitably composed and manufactured HIPE foam absorbent layer as described herein, for example, has a greater affinity for menstrual fluid than such a topsheet, and thereby, has the capability to draw and retain fluid away from the topsheet when the two are disposed and held in sufficiently effective proximate, contacting relationship with each other. When the absorbent layer has a sufficient volume, it can serve this function over a reasonably suitable time of use of the pad.

A topsheet material having a relatively flat outward-facing (lower) surface as described above, may be more reliably bonded to a subjacent foam layer, via a discontinuous pattern of adhesive, than a topsheet material having a relatively more textured outward-facing surface. Thus, the combination of a topsheet material having the features described above, with a pattern of adhesive bonding as described above, provides synergy of features to enable more effective fluid acquisition, while providing a pleasing visibly-textured wearer-facing (upper) surface.

### Backsheet

The backsheet 30 may be positioned adjacent an outward-facing surface of the absorbent layer 40 and may be joined thereto by any suitable attachment methods. For example, the backsheet 30 may be secured to the absorbent layer 40 by a uniform continuous layer of adhesive, a patterned layer of adhesive, or an array of separate lines, spirals, or spots of adhesive. Alternatively, the attachment method may include heat bonds, pressure bonds, ultrasonic bonds, dynamic mechanical bonds, or any other suitable attachment mechanisms or combinations thereof. In other examples, it is contemplated that the absorbent layer 40 is not joined directly to the backsheet 30.

The backsheet 30 may be impervious, or substantially impervious, to liquids (e.g., urine, menstrual fluid) and may be manufactured from a thin plastic film, although other flexible liquid impervious materials may also be used. As used herein, the term “flexible” refers to materials which are compliant and will readily conform to the general shape and contours of the human body. The backsheet 30 may prevent, or at least substantially inhibit, fluids absorbed and contained within the absorbent layer 40 from escaping and reaching articles of the wearer's clothing which may contact the feminine hygiene pad 10 such as underpants and outer clothing. However, in some instances, the backsheet 30 may be made and/or adapted to permit vapor to escape from the absorbent layer 40 (i.e., the backsheet is made to be breathable), while in other instances the backsheet 30 may be made so as not to permit vapors to escape (i.e., it is made to be non-breathable). Thus, the backsheet 30 may comprise a polymeric film such as thermoplastic films of polyethylene or polypropylene. A suitable material for the backsheet 30 is a thermoplastic film having a thickness of from about 0.012 mm (0.5 mil) to about 0.051 mm (2.0 mils), for example. Any suitable backsheet known in the art may be utilized with the present invention.

Some suitable examples of backsheets are described in U.S. Pat. Nos. 5,885,265; 4,342,314; and 4,463,045. Suitable single layer breathable backsheets for use herein include those described for example in GB A 2184 389; GB A 2184 390; GB A 2184 391; U.S. Pat. Nos. 4,591,523, 3,989,867, 3,156,242; WO 97/24097; U.S. Pat. Nos. 6,623,464; 6,664,439 and 6,436,508.

The backsheet may have two layers: a first layer comprising a vapor permeable aperture-formed film layer and a second layer comprising a breathable microporous film layer, as described in U.S. Pat. No. 6,462,251. Other suitable examples of dual or multi-layer breathable backsheets for use herein include those described in U.S. Pat. Nos. 3,881,489, 4,341,216, 4,713,068, 4,818,600; EP 203 821, EP 710 471; EP 710 472, and EP 0 793 952.

### Packages of Absorbent Articles

A package of absorbent articles may include two or more absorbent articles, the absorbent articles including a liquid permeable topsheet, a liquid impervious backsheet, and an absorbent layer positioned therebetween. A thickness of a portion of the absorbent layer of at least one absorbent article in the package may be from about 0 mm to about 4 mm, from about 0.5 mm to about 4 mm, from about 1 mm to about 4 mm, from about 2 mm to about 4 mm, from about 3 mm to about 4 mm, from about 0 mm to about 3 mm, from about 0.5 mm to about 3 mm, from about 1 mm to about 3 mm, from about 2 mm to about 3 mm, from about 0 mm to about 2 mm, from about 0.5 mm to about 2 mm, from about 1 mm to about 2 mm, or from about 0.5 mm to about 1 mm greater than a thickness of a portion of the absorbent layer of another absorbent article in the package. The absorbent layer of at least one absorbent article in the package may have a thickness at least 15 percent greater, at least 20 percent greater, or at least 25 percent greater than the absorbent layer of at least one other absorbent article in the same package.

The standard deviation between the thickness of a portion of the absorbent layer of at least one absorbent article to the thickness of a portion of the absorbent layer of at least one other absorbent article in the package may be from about 0.01 mm to about 0.5 mm, from about 0.01 mm to about 0.4 mm, from about 0.01 mm to about 0.3 mm, from about 0.01 mm to about 0.25 mm, from about 0.01 mm to about 0.2 mm, from about 0.01 mm to about 0.1 mm, from about 0.01 mm to about 0.05 mm, from about 0.05 mm to about 0.5 mm, from about 0.05 mm to about 0.4 mm, from about 0.05 mm to about 0.3 mm, from about 0.05 mm to about 0.25 mm, from about 0.05 mm to about 0.2 mm, from about 0.05 mm to about 0.1 mm, from about 0.1 mm to about 0.5 mm, from about 0.1 mm to about 0.4 mm, from about 0.1 mm to about 0.3 mm, from about 0.1 mm to about 0.25 mm, from about 0.1 mm to about 0.2 mm, from about 0.1 mm to about 0.1 mm, from about 0.2 mm to about 0.5 mm, from about 0.2 mm to about 0.4 mm, from about 0.2 mm to about 0.3 mm, from about 0.2 mm to about 0.25 mm, from about 0.2 mm to about 0.2 mm, from about 0.25 mm to about 0.5 mm, from about 0.25 mm to about 0.4 mm, from about 0.25 mm to about 0.3 mm, from about 0.3 mm to about 0.5 mm, from about 0.3 mm to about 0.4 mm, or from about 0.4 mm to about 0.5 mm.

A portion of an absorbent layer of an absorbent article may have a thickness of from about 0 mm to 4 mm, from about 0.5 mm to about 4 mm, from about 1 mm to about 4 mm, from about 2 mm to about 4 mm, from about 3 mm to about 4 mm, from about 0 mm to about 3 mm, from about 0.5 mm to about 3 mm, from about 1 mm to about 3 mm, from about 2 mm to about 3 mm, from about 0 mm to about 2 mm, from about 0.5 mm to about 2 mm, from about 1 mm to about 2 mm, or from about 0.5 mm to about 1 mm greater than the thickness of a portion of an absorbent layer of another absorbent article in a different package, wherein the different package includes two or more absorbent articles. The different package may contain absorbent articles that are marketed to have the same size, shape, and absorbency as the absorbent articles in the first package, and may be produced by the same manufacturer as the absorbent articles in the first package, but may be from the same manufacturing lot or a different manufacturing lot. The two or more absorbent articles of the different package may include a liquid permeable topsheet, a liquid impervious backsheet, and an absorbent layer positioned therebetween.

The standard deviation between the thickness of a portion of the absorbent layer of at least one absorbent article in a package to the thickness of a portion of the absorbent layer of at least one absorbent article in a different package may be from about 0.01 mm to about 0.5 mm, from about 0.01 mm to about 0.4 mm, from about 0.01 mm to about 0.3 mm, from about 0.01 mm to about 0.25 mm, from about 0.01 mm to about 0.2 mm, from about 0.01 mm to about 0.1 mm, from about 0.01 mm to about 0.05 mm, from about 0.05 mm to about 0.5 mm, from about 0.05 mm to about 0.4 mm, from about 0.05 mm to about 0.3 mm, from about 0.05 mm to about 0.25 mm, from about 0.05 mm to about 0.2 mm, from about 0.05 mm to about 0.1 mm, from about 0.1 mm to about 0.5 mm, from about 0.1 mm to about 0.4 mm, from about 0.1 mm to about 0.3 mm, from about 0.1 mm to about 0.25 mm, from about 0.1 mm to about 0.2 mm, from about 0.1 mm to about 0.1 mm, from about 0.2 mm to about 0.5 mm, from about 0.2 mm to about 0.4 mm, from about 0.2 mm to about 0.3 mm, from about 0.2 mm to about 0.25 mm, from about 0.2 mm to about 0.2 mm, from about 0.25 mm to about 0.5 mm, from about 0.25 mm to about 0.4 mm, from about 0.25 mm to about 0.3 mm, from about 0.3 mm to about 0.5 mm, from about 0.3 mm to about 0.4 mm, or from about 0.4 mm to about 0.5 mm.

### Methods of Producing an Absorbent Article

Methods of producing absorbent articles may include obtaining a liquid permeable topsheet, a liquid impervious backsheet, and an absorbent layer, wherein the topsheet includes a fibrous nonwoven material, an upper topsheet surface and a lower topsheet surface, and a first portion adjacent the upper topsheet surface and a second portion adjacent the lower topsheet surface. Methods of producing absorbent articles may further include applying adhesive to either the topsheet, the absorbent layer, or a combination thereof. Methods of producing absorbent articles may further include compressing the topsheet and the absorbent layer with a pressure, wherein the pressure is selected so that the topsheet is at least partially attached to the absorbent layer using an adhesive, wherein the adhesive does not reach the first topsheet portion. In some examples, the pressure may be varied based on the variation in thickness of the absorbent layer. The pressure may be adjusted using pressure sensing technology, using gap varying technology, or using other techniques or technology that would be known to those of ordinary skill in the art.

Without being bound by theory, it is believed that even minor variations in the thickness of the absorbent core can contribute to forming the textural irregularities. When pressure is applied by passing a topsheet and an absorbent layer through a gap, the pressure that the topsheet and absorbent layer experience can vary with the variation in the absorbent layer thickness. This variation in absorbent layer thickness may cause adhesive to bleed through to the first topsheet portion in areas where the absorbent layer is thicker, and other areas where the absorbent layer is thinner may not be compressed at all and may have poor adhesion. However, when it is ensured that the pressure on the topsheet and the absorbent layer is adjusted based on the thickness of the absorbent layer so that adhesive does not penetrate into the first topsheet portion, textural irregularities are minimized. The pressure needed to compress the topsheet and the absorbent layer so that adhesive does not penetrate the first topsheet layer may vary based on the thickness of the topsheet and the thickness of the absorbent layer.

### Measurement Methods

### Surface Topography Method for Sdcₜ

In the Surface Topography measurement method, the areal surface topology of a nonwoven web sample surface is measured using optical profilometry. The 3D surface data are then processed and analyzed to extract the microscale areal surface roughness parameter Sq (root mean square height), the surface complexity parameter Sdr (Developed Area), and the core height parameter Sdcₜ. All sample preparation and testing are performed in a conditioned room maintained at 23° C.±2° C. and 50%±2% relative humidity, and prepared samples are kept in this environment for at least 24 hours prior to measurement.

### Preparation

To prepare a sample of nonwoven web material to be obtained from finished feminine hygiene pad, the topsheet layer is removed from an absorbent article exposing the underlying absorbent layer. The topsheet layer is carefully removed in a manner that avoids distortion of the surface topography of the upper and lower surfaces of the material. A cryogenic spray (such as CYTO-FREEZE, Control Company, Houston Texas, or equivalent) may be used to facilitate clean separation of the topsheet material from the underlying absorbent layer and avoid tearing of the upper foam surface. Samples of topsheet materials with any tears or residua of folds should not be used. Five replicate samples are prepared for testing.

### 3D Surface Image Acquisition

A three-dimensional (3D) surface topography image of the upper and lower surfaces of the topsheet sample and the upper surface of the absorbent layer of the sample pad is obtained using a DLP-based, structured-light 3D surface topography measurement system (a suitable surface topography measurement system is the MikroCAD Premium instrument commercially available from LMI Technologies Inc., Vancouver, Canada, or equivalent). The system includes the following main components: a) a Digital Light Processing (DLP) projector with direct digital controlled micro-mirrors; b) a CCD camera with at least a 1600×1200 pixel resolution; c) projection optics adapted to a measuring area of at least 140 mm×105 mm; d) recording optics adapted to a measuring area of 140 mm×105 mm; e) a table tripod based on a small hard stone plate; f) a blue LED light source; g) a measuring, control, and evaluation computer running surface texture analysis software (a suitable software is MikroCAD software with MountainsMap technology, or equivalent); and h) calibration plates for lateral (XY) and vertical (Z) calibration available from the vendor.

The optical 3D surface topography measurement system measures the surface height of a sample using the digital micro-mirror pattern fringe projection technique. The result of the measurement is a 3D data set of surface height (defined as the Z-axis) versus displacement in the horizontal (XY) plane. This 3D data set can also be thought of as an image in which every pixel in the image has an associated XY displacement, and the value of the pixel is the recorded Z-axis height value. The system has a field of view of 140×105 mm with an XY pixel resolution of approximately 85 microns, and a height resolution of 0.5 microns, with a total possible height range of 32 mm.

The instrument is calibrated according to manufacturer's specifications using the calibration plates for lateral (XY plane) and vertical (Z-axis) available from the vendor.

The sample is placed flat on the table beneath the camera. The sample may be very gently pulled taut (not to stretching) along X and Y dimensions to flatten out any large-scale waviness, and weights may be placed on the sample outside of the measurement area to hold it taut. A 3D surface topology image of the sample surface is collected by following the instrument manufacturer's recommended measurement procedures, which may include focusing the measurement system and performing a brightness adjustment. No pre-filtering options are used. The collected height image file is saved to the evaluation computer running the surface texture analysis software.

### 3D Surface Image Analysis

Analysis of a surface height image is initiated by opening the image in the surface texture analysis software. A recommended filtration process is described in ISO 25178-2:2021. Accordingly, the following filtering procedure is performed on each image: 1) If the sample surface is smaller than the image field of view, select the largest rectangular region of interest that can fit within the sample surface and crop the image to that size; 2) a Gaussian low pass S-filter with a nesting index (cut-off) of 2 mm to remove short scale components; 3) an F-operation of removing the least squares plane to level the surface; and 4) a Robust Gaussian high pass L-filter with a nesting index (cut-off) of 25 mm (ISO 16610-71) to remove long scale components. Both Gaussian filters are run utilizing end effect correction. This filtering procedure produces the S-L surface from which the areal surface texture parameters will be calculated.

This filtering procedure produces the surface from which the Sq (root mean square) values, as described in ISO 25178-2:2021, are calculated. Record the surface roughness values for Sq to the nearest 0.01 mm. This procedure is repeated for the remaining replicate samples. Average together the 5 replicate Sq measures and report these values to the nearest 0.01 mm.

The parameter Sdr, as described in ISO 25178-2:2021, is calculated on the filtered 3D topography image and recorded. Sdr is the ratio of the actual surface area of the sample to the projected horizonal area of the image and is given in percent (%). Record the surface complexity values for Sdr to the nearest 0.01%. This procedure is repeated for the remaining replicate samples. Average together the 5 replicate Sdr measures and report these values to the nearest 0.01%.

The core height value, Sdcₜ, as described in ISO 25178-2:2021, is derived from the Areal Material Ratio (Abbott-Firestone) curve, which is the cumulative curve of the surface height distribution histogram versus the range of surface heights. The core height value is the height difference between the material ratios Smr1 and Smr2 as read off the Areal Material Ratio curve. Smr1, set to 5%, is the material ratio which separates the protruding peaks from the core roughness region. Smr2, set to 95%, is the material ratio which separates the deep valleys from the core roughness region. Record the surface height Sdcₜ value to the nearest 0.01 mm. Average together the five replicate Sdcₜ values and report to the nearest 0.01 mm.

### Unstructured Region Surface Topography Method for Sdcₐ

In the Unstructured Region Surface Topography measurement method, the areal surface topology of an unstructured region of an absorbent article sample surface is measured using optical profilometry. The 3D surface data are then processed and analyzed to extract the core height parameter Sdcₐ. All sample preparation and testing are performed in a conditioned room maintained at 23° C.±2° C. and 50%±2% relative humidity, and prepared samples are kept in this environment for at least 24 hours prior to measurement.

### Sample Preparation

An absorbent article test sample is prepared by removing it from any outer wrapping and unfolding it fully to expose the textured topsheet on the upper body facing surface. Five replicate samples are prepared for testing.

### 3D Surface Image Acquisition

A three-dimensional (3D) surface topography image of the textured surface of the topsheet on the sample absorbent article is obtained using a DLP-based, structured-light 3D surface topography measurement system (a suitable surface topography measurement system is the MikroCAD Premium instrument commercially available from LMI Technologies Inc., Vancouver, Canada, or equivalent). The system includes the following main components: a) a Digital Light Processing (DLP) projector with direct digital controlled micro-mirrors; b) a CCD camera with at least about a 1300×1000 pixel resolution; c) projection optics adapted to a measuring area of at least about 25 mm×20 mm; d) recording optics adapted to a measuring area of about 25 mm×20 mm; e) a table tripod based on a small hard stone plate; f) a blue LED light source; g) a measuring, control, and evaluation computer running surface texture analysis software (a suitable software is ODSCAD MikroCAD software with MountainsMap technology, or equivalent); and h) calibration plates for lateral (XY) and vertical (Z) calibration available from the vendor.

The optical 3D surface topography measurement system measures the surface height of a sample using the digital micro-mirror pattern fringe projection technique. The result of the measurement is a 3D data set of surface height (defined as the Z-axis) versus displacement in the horizontal (XY) plane. This 3D data set can also be thought of as an image in which every pixel in the image has an associated XY displacement, and the value of the pixel is the recorded Z-axis height value. The system has a field of view of 25×20 mm with an XY pixel resolution of approximately 19 microns, and a height resolution of at least 0.5 microns, with a total possible height range of 8 mm.

The instrument is calibrated according to manufacturer's specifications using the calibration plates for lateral (XY plane) and vertical (Z-axis) available from the vendor.

The absorbent article sample is placed flat on the table beneath the camera with the textured topsheet on the upper body facing surface oriented upward toward the camera. The sample may be gently pulled taut (not to stretching) along X and Y dimensions to flatten out any large-scale waviness, and weights may be placed on the sample outside of the measurement area to hold it taut. An unstructured region of the sample surface is selected for imaging. The image field of view of the selected unstructured region should include portions of adjacent structured regions which define the perimeter or boundary surrounding the selected unstructured region. The selected region should not include any creases or residua of folds. For example, referring again to FIG. 7, the unstructured region 920 selected for imaging should include the adjacent portions of structured regions 910. Thus, the selected unstructured region is defined by boundary line 915.

A 3D surface topology image of the sample surface is collected by following the instrument manufacturer's recommended measurement procedures, which may include focusing the measurement system and performing a brightness adjustment. No pre-filtering options are used. The collected height image file is saved to the evaluation computer running the surface texture analysis software.

### 3D Surface Image Analysis

Analysis of a surface height image is initiated by opening the image in the surface texture analysis software. A recommended filtration process is described in ISO 25178-2:2021. Accordingly, the following filtering procedure is performed on each image: 1) an F-operation of removing the least squares plane to level the surface; 2) Manually drawing a region of interest that includes the selected unstructured region and a portion of the adjacent structured region such that the total area of the individual bond points, attenuated regions, apertures, or combinations thereof of the included structured region equates to approximately 5% of the selected region of interest area and defines the perimeter or boundary of the unstructured region (For example, see FIG. 7, where the unstructured region is defined by boundary line 915) and 3) Cropping or extracting the selected region of interest. This filtering procedure produces the surface from which the following areal surface texture parameters will be calculated.

The core height value, Sdcₐ, as described in ISO 25178-2:2021, is derived from the Areal Material Ratio (Abbott-Firestone) curve, which is the cumulative curve of the surface height distribution histogram versus the range of surface heights. The core height value is the height difference between the material ratios Smr1 and Smr2 as read off the Areal Material Ratio curve. Smr1, set to 5%, is the material ratio which separates the protruding peaks from the core roughness region. Smr2, set to 95%, is the material ratio which separates the deep valleys from the core roughness region. Record the surface height Sdcₐ value to the nearest 0.01 mm. Average together the five replicate Sdcₐ values and report to the nearest 0.01 mm.

### Adhesive Penetration Via SEM Method

A Scanning Electron Microscope (SEM) is used to obtain images of the cross-section of a test specimen to enable visualization of the microstructure of and the connectivity between the layers within an absorbent article, including the depth that an adhesive has penetrated into a given layer. All testing is performed in a room maintained at a temperature of 23° C.±2.0° C. and a relative humidity of 50%±2% and samples are conditioned under the same environmental conditions for at least 2 hours prior to testing.

Secondary Electron (SE) images are obtained using an SEM such as the FEI Quanta 450 (available from FEI Company, Hillsboro, OR), or equivalent. The instrument is calibrated according to the manufacturer's instructions prior to use to ensure an accurate distance scale.

A sub-sample is obtained by removing it from an absorbent article test sample as follows. The subsample is taken from an area free of folds or wrinkles using care to not impart any contamination or distortion to the region that will be analyzed. The subsample is about 3.5 cm wide (parallel to the lateral axis of the absorbent article test sample) by about 2.0 cm long (parallel to the longitudinal axis of the absorbent article test sample), and it includes all of the layers within the thickness of the article (i.e., topsheet to backsheet). A cross-sectioned test specimen is prepared by submerging the subsample in liquid nitrogen and fracturing an edge along the subsample's width with a fresh, new razor blade (such as VWR Single Edge Industrial Razor blade No. 9, surgical carbon steel, or equivalent). Further preparation of the test specimen is decided upon by the analyst skilled in the art, and this might include staining (e.g., osmium or sputter-coating) to enhance the contrast between the adhesive and the material layer it has been applied to.

The cross-sectioned test specimen is adhered to the SEM mount so that the cross-section can be viewed and the specific layer and region of interest can be identified. A sufficient number of high resolution images (e.g., 6.8 megapixel) are obtained to in order to represent at least ten replicate regions of interest.

Quantitative measures within the collection of images are made using image analysis software (e.g., built into the SEM instrument, or standalone software such as ImageJ v. 1.52, National Institute of Health, USA, or equivalent). One such quantitative measure is the penetration depth of the adhesive into the topsheet layer. Using the image analysis tools, the thickness of the topsheet layer is measured within the distance calibrated image and recorded to the nearest 0.1 micron. The thickness of the adhesive that has penetrated into the topsheet layer is measured and recorded to the nearest 0.1 micron. Percent penetration of the adhesive into the topsheet is calculated by dividing the thickness of the adhesive by the thickness of the topsheet and then multiplying by 100 and recording to the nearest 1 percent. In like fashion, the percent penetration is obtained for at least ten replicate regions of interest within the collection of images. The arithmetic mean of percent penetration is calculated across the ten replicate measurements and reported as percent penetration into the topsheet to the nearest 1 percent.

In like fashion, percent penetration of the adhesive into other layers within the absorbent article (e.g., the absorbent core) can also be obtained and reported.

### Thickness Test

The thickness of test sample is measured as the distance between a reference platform on which the test sample rests and a pressure foot that exerts a specified amount of pressure onto the sample over a specified amount of time. Thickness is measured on an absorbent article that includes all layers within the article. Additionally, thickness can be measured on an individual material layer excised from an absorbent article (e.g., the absorbent layer). All measurements are performed in a laboratory maintained at 23° C.±2° C. and 50%±2% relative humidity and test samples are conditioned in this environment for at least 2 hours prior to testing.

The thickness of the test sample is measured using a manually operated micrometer equipped with a pressure foot capable of exerting a steady pressure of 1.25 kPa±0.05 kPa (0.18 psi) onto the test location on the test sample. The manually operated micrometer is a dead weight type instrument with readings accurate to 0.01 mm. A suitable instrument is Mitutoyo Series 543 ID-C Digimatic, available from VWR International, or equivalent. The pressure foot is a smooth flat circular movable face with a diameter that is 17 mm. The test sample is supported by a horizontal flat reference platform that is larger than and parallel to the surface of the pressure foot. The system is calibrated and operated per the manufacturer's instructions.

To measure the thickness of an absorbent article, the test sample is prepared by removing the absorbent article from any outer packaging, and if the article is folded, gently unfold it. The protective layer (i.e., release paper) covering the garment attachment adhesive is removed and a light dusting of talc is applied to mitigate tackiness. To measure the thickness of an individual material layer (e.g., the absorbent layer), the layer is excised from the absorbent article using care to prevent any stretching or other distortion to the material. If the layer contains adhesive residue, a light dusting of talc can be applied to mitigate tackiness. In like fashion, a minimum of ten replicate test samples of the same production date are prepared, with the ideal number of replicates being equal to the number of absorbent articles contained within a given package.

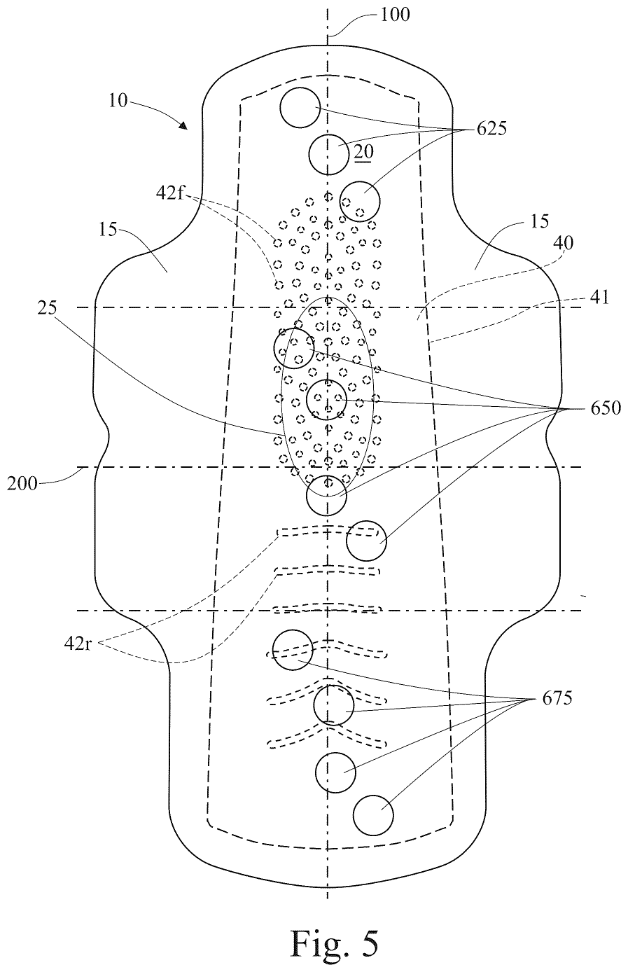

The thickness of the test sample is measured at the maximum number of non-overlapping test location across the body-facing side of the article (or material layer) such that the thickness of the article (or material layer) at the front, middle, and rear, as well as left and right sides, are thoroughly represented. Test locations exclude regions where there is any residual of fold lines. When measuring the thickness of an absorbent article, regions where all layers are not present (i.e., the wings and the outermost edges of the test sample) are also excluded. FIG. 5 depicts a set of test locations within an example article.

To measure thickness, the micrometer is zeroed against the horizontal flat reference platform. The test sample is then placed on the platform with the first test location centered below the pressure foot. The pressure foot is gently lowered using a descent rate of 3.0 mm±1.0 mm per second until the full pressure is exerted onto the test sample. The full pressure is applied to the test sample for 5 seconds and then the thickness of the test sample is recorded to the nearest 0.01 mm for that test location. In like fashion, the procedure is repeated until the thickness of all test locations are measured. In like fashion, the procedure is then repeated for all of the replicate test samples.

The arithmetic mean (μ) and standard deviation (SD) amongst groupings of thickness measurements can be calculated to compare variability at test locations within a given test sample, and to compare variability of test locations between replicate test samples.

The arithmetic mean (μ) of thickness is calculated using the following equation, and reported to the nearest 0.01 mm:

$$μ = \frac{\sum x}{N}$$

where Σ means “sum of”, x is an individual thickness value in the data set that includes all test locations within the group to be compared, and N is the total number of data points within the group to be compared.

The standard deviation (SD) of thickness is calculated using the following equation, and reported to the nearest 0.01 mm:

$$SD = \sqrt{\frac{\sum {\vert x - μ \vert}^{2}}{N}}$$

where Σ means “sum of”, x is an individual thickness value in the data set that includes all test locations within the group to be compared, μ is the reported arithmetic mean of thickness amongst the group to be compared, and N is the total number of data points within the group to be compared.

### Absorbent Article Basis Weight Method

Basis weights of materials described herein may be determined by several available techniques, but a simple representative technique involves taking an absorbent article or other consumer product, removing any elastic which may be present and stretching the absorbent article or other consumer product to its full length. A punch die having an area of 45.6 cm² is then used to cut a piece of the material to be analyzed (e.g., entire absorbent article, topsheet, backsheet, etc.) from the approximate center of the absorbent article in a location which avoids to the greatest extent possible any adhesive which may be used to fasten the material to any other layers which may be present and removing the material from other layers (using cryogenic spray, such as Cyto-Freeze, Control Company, Houston, Tex., if needed). The sample is then weighed to the nearest 0.1 g, and dividing by the area of the punch die yields the basis weight of the material. Results are reported as a mean of 5 samples to the nearest 0.1 g/cm².

### Thickness-Pressure Method

The thickness of a test specimen is measured as the distance between a reference platform on which the specimen rests and a pressure foot that exerts a specified amount of pressure onto the specimen over a specified amount of time. All measurements are performed in a laboratory maintained at 23° C.±2° C. and 50%±2% relative humidity and test specimens are conditioned in this environment for at least 2 hours prior to testing.

Thickness is measured with a manually-operated micrometer equipped with a pressure foot capable of exerting a steady pressure (7 g/cm²) onto the test specimen. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat ground circular movable face with a diameter that is smaller than the test specimen and capable of exerting the required pressure. A suitable pressure foot has a diameter of 25.4 mm, however a smaller or larger foot can be used depending on the size of the specimen being measured. The test specimen is supported by a horizontal flat reference platform that is larger than and parallel to the surface of the pressure foot. The system is calibrated and operated per the manufacturer's instructions.

Measurements are made on test specimens taken from rolls or sheets of the raw material, or test specimens obtained from a material layer removed from an absorbent article. When excising the material layer from an absorbent article, use care to not impart any contamination or distortion to the layer during the process. The excised layer should be free from residual adhesive and any fibers that may have transferred from underlying layers. To ensure that all adhesive and any transferred fibers are removed, soak the layer in a suitable solvent that will dissolve the adhesive and release any transferred fibers present without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained from an area free of folds or wrinkles, and it must be larger than the pressure foot.

To measure thickness at a confining pressure of 7 g/cm², first zero the micrometer against the horizontal flat reference platform. Place the test specimen on the platform with the test location centered below the pressure foot. Gently lower the pressure foot with a descent rate of 3.0 mm+1.0 mm per second until the full pressure is exerted onto the test specimen. Wait 5 seconds and then record the thickness of the test specimen to the nearest 0.01 mm. In like fashion, repeat for a total of ten replicate test specimens. Calculate the arithmetic mean for all thickness measurements obtained at a confining pressure of 7 g/cm² and report as Thickness at 7 g/cm² to the nearest 0.01 mm.

### EXAMPLES

The following Examples are offered by way of illustration and are presented in a manner such that one skilled in the art should recognize are not meant to be limiting to the present disclosure as a whole or to the appended claims.

### Example 1

A box of 10 ALWAYS INFINITY brand size F3 (270 mm length) feminine hygiene pads from a first manufacturing lot manufactured by The Procter & Gamble Company, Cincinnati, Ohio, was obtained. The absorbent layer was made of HIPE foam. Three samples, 625, from the front region of the pad, four samples, 650, from the middle region of the pad, and four samples, 675, from the rear region of the pad were obtained, as shown in FIG. 5. The samples, which included the topsheet, absorbent layer, and backsheet, were measured to determine thickness according to the Thickness test. Variations in thickness were believed to be caused by variations in the thickness of the absorbent layer, as the topsheet and backsheet thicknesses were believed to be constant. The results were as shown in Tables 4-7 and measurements are reported in millimeters (mm).

TABLE 4
Average, Stan Dev, Min, and Max of 3 Points of the Pad Front

| Pad # | f1 | f3 | f5 | Average | Std Dev | MIN | MAX |
|---|---|---|---|---|---|---|---|
| 1 | 2.42 | 2.44 | 2.44 | 2.43 | 0.01 | 2.42 | 2.44 |
| 2 | 2.40 | 2.34 | 2.40 | 2.38 | 0.03 | 2.34 | 2.40 |
| 3 | 2.36 | 2.35 | 2.47 | 2.39 | 0.07 | 2.35 | 2.47 |
| 4 | 2.40 | 2.41 | 2.39 | 2.40 | 0.01 | 2.39 | 2.41 |
| 5 | 2.35 | 2.36 | 2.41 | 2.37 | 0.03 | 2.35 | 2.41 |
| 6 | 2.43 | 2.41 | 2.44 | 2.43 | 0.02 | 2.41 | 2.44 |
| 7 | 2.39 | 2.35 | 2.46 | 2.40 | 0.06 | 2.35 | 2.46 |
| 8 | 2.44 | 2.38 | 2.45 | 2.42 | 0.04 | 2.38 | 2.45 |
| 9 | 2.35 | 2.36 | 2.40 | 2.37 | 0.03 | 2.35 | 2.40 |
| 10 | 2.37 | 2.45 | 2.41 | 2.41 | 0.04 | 2.37 | 2.45 |

TABLE 5
Average, Stan Dev, Min, and Max of 4 Points of the Pad Middle

| Pad # | m1 | m3 | m6 | m8 | Average | Std Dev | MIN | MAX |
|---|---|---|---|---|---|---|---|---|
| 1 | 2.53 | 2.42 | 2.39 | 2.56 | 2.48 | 0.08 | 2.39 | 2.56 |
| 2 | 2.47 | 2.41 | 2.43 | 2.62 | 2.48 | 0.10 | 2.41 | 2.62 |
| 3 | 2.35 | 2.48 | 2.30 | 2.37 | 2.38 | 0.08 | 2.30 | 2.48 |
| 4 | 2.64 | 2.41 | 2.44 | 2.60 | 2.52 | 0.11 | 2.41 | 2.64 |
| 5 | 2.49 | 2.33 | 2.41 | 2.68 | 2.48 | 0.15 | 2.33 | 2.68 |
| 6 | 2.63 | 2.40 | 2.45 | 2.57 | 2.51 | 0.11 | 2.40 | 2.63 |
| 7 | 2.57 | 2.36 | 2.52 | 2.46 | 2.48 | 0.09 | 2.36 | 2.57 |
| 8 | 2.58 | 2.34 | 2.42 | 2.51 | 2.46 | 0.10 | 2.34 | 2.58 |
| 9 | 2.44 | 2.37 | 2.37 | 2.64 | 2.46 | 0.13 | 2.37 | 2.64 |
| 10 | 2.54 | 2.39 | 2.28 | 2.74 | 2.49 | 0.20 | 2.28 | 2.74 |

TABLE 6
Average, Stan Dev, Min, and Max of 4 Points of the Pad Rear

| Pad # | r1 | r3 | r6 | r8 | Average | Std Dev | MIN | MAX |
|---|---|---|---|---|---|---|---|---|
| 1 | 2.46 | 2.60 | 2.55 | 2.39 | 2.50 | 0.09 | 2.39 | 2.60 |
| 2 | 2.45 | 2.35 | 2.30 | 2.37 | 2.37 | 0.06 | 2.30 | 2.45 |
| 3 | 2.57 | 2.44 | 2.58 | 2.38 | 2.49 | 0.10 | 2.38 | 2.58 |
| 4 | 2.50 | 2.40 | 2.33 | 2.37 | 2.40 | 0.07 | 2.33 | 2.50 |
| 5 | 2.49 | 2.40 | 2.35 | 2.42 | 2.42 | 0.06 | 2.35 | 2.49 |
| 6 | 2.42 | 2.47 | 2.36 | 2.37 | 2.41 | 0.05 | 2.36 | 2.47 |
| 7 | 2.60 | 2.58 | 2.42 | 2.39 | 2.50 | 0.11 | 2.39 | 2.60 |
| 8 | 2.36 | 2.64 | 2.53 | 2.36 | 2.47 | 0.14 | 2.36 | 2.64 |
| 9 | 2.46 | 2.64 | 2.42 | 2.34 | 2.47 | 0.13 | 2.34 | 2.64 |
| 10 | 2.48 | 2.58 | 2.36 | 2.37 | 2.45 | 0.10 | 2.36 | 2.58 |

TABLE 7
Entire Package Average, Stan Dev, Min, and Max of the Pad

| | f1 | f3 | f5 | m1 | m3 | m6 | m8 | r1 | r3 | r6 | r8 |
|---|---|---|---|---|---|---|---|---|---|---|---|
| Entire Pack | 2.39 | 2.39 | 2.43 | 2.52 | 2.39 | 2.40 | 2.58 | 2.48 | 2.51 | 2.42 | 2.38 |
| Average: | | | | | | | | | | | |
| Entire | 0.03 | 0.04 | 0.03 | 0.09 | 0.04 | 0.07 | 0.11 | 0.07 | 0.11 | 0.10 | 0.02 |
| Pack Std | | | | | | | | | | | |
| Dev: | | | | | | | | | | | |
| Entire Pack | 2.35 | 2.34 | 2.39 | 2.35 | 2.33 | 2.28 | 2.37 | 2.36 | 2.35 | 2.30 | 2.34 |
| MIN: | | | | | | | | | | | |
| Entire Pack | 2.44 | 2.45 | 2.47 | 2.64 | 2.48 | 2.52 | 2.74 | 2.60 | 2.64 | 2.58 | 2.42 |
| MAX: | | | | | | | | | | | |

### Example 2

A box of 10 ALWAYS INFINITY brand size F3 (270 mm length) feminine hygiene pads from a second manufacturing lot manufactured by The Procter & Gamble Company, Cincinnati, Ohio, was obtained. The absorbent layer was made of HIPE foam. Three samples, 625, from the front region of the pad, four samples, 650, from the middle region of the pad, and four samples, 675, from the rear region of the pad were obtained, as shown in FIG. 5. The samples, which included the topsheet, absorbent layer, and backsheet, were measured to determine thickness according to the Thickness test. Variations in thickness were believed to be caused by variations in the thickness of the absorbent layer, as the topsheet and backsheet thicknesses were believed to be constant. The results were as shown in Tables 8-11 and measurements are reported in millimeters (mm).

TABLE 8
Average, Stan Dev, Min, and Max of 3 Points of the Pad Front

| Pad # | f1 | f3 | f5 | Average | Std Dev | MIN | MAX |
|---|---|---|---|---|---|---|---|
| 1 | 2.58 | 2.97 | 3.18 | 2.91 | 0.30 | 2.58 | 3.18 |
| 2 | 3.04 | 3.11 | 3.33 | 3.16 | 0.15 | 3.04 | 3.33 |
| 3 | 2.60 | 3.03 | 3.10 | 2.91 | 0.27 | 2.60 | 3.10 |
| 4 | 2.87 | 2.94 | 3.41 | 3.07 | 0.29 | 2.87 | 3.41 |
| 5 | 2.70 | 3.08 | 3.12 | 2.97 | 0.23 | 2.70 | 3.12 |
| 6 | 2.88 | 2.93 | 3.46 | 3.09 | 0.32 | 2.88 | 3.46 |
| 7 | 2.66 | 2.77 | 3.16 | 2.86 | 0.26 | 2.66 | 3.16 |
| 8 | 2.90 | 2.93 | 3.24 | 3.02 | 0.19 | 2.90 | 3.24 |
| 9 | 2.70 | 2.81 | 3.12 | 2.88 | 0.22 | 2.70 | 3.12 |
| 10 | 3.03 | 3.00 | 3.33 | 3.12 | 0.18 | 3.00 | 3.33 |

TABLE 9
Average, Stan Dev, Min, and Max of 4 Points of the Pad Middle

| Pad # | m1 | m3 | m6 | m8 | Average | Std Dev | MIN | MAX |
|---|---|---|---|---|---|---|---|---|
| 1 | 3.09 | 2.62 | 3.12 | 3.12 | 2.99 | 0.25 | 2.62 | 3.12 |
| 2 | 3.25 | 2.56 | 3.10 | 3.12 | 3.01 | 0.31 | 2.56 | 3.25 |
| 3 | 3.20 | 2.73 | 2.72 | 3.20 | 2.96 | 0.27 | 2.72 | 3.20 |
| 4 | 3.22 | 2.69 | 2.68 | 3.17 | 2.94 | 0.30 | 2.68 | 3.22 |
| 5 | 3.17 | 2.62 | 2.82 | 3.12 | 2.93 | 0.26 | 2.62 | 3.17 |
| 6 | 3.24 | 2.60 | 3.23 | 3.09 | 3.04 | 0.30 | 2.60 | 3.24 |
| 7 | 3.20 | 2.62 | 3.18 | 3.18 | 3.05 | 0.28 | 2.62 | 3.20 |
| 8 | 3.23 | 2.57 | 3.33 | 3.07 | 3.05 | 0.34 | 2.57 | 3.33 |
| 9 | 3.17 | 2.75 | 3.17 | 3.23 | 3.08 | 0.22 | 2.75 | 3.23 |
| 10 | 3.25 | 2.80 | 3.12 | 3.16 | 3.08 | 0.20 | 2.80 | 3.25 |

TABLE 10
Average, Stan Dev, Min, and Max of 4 Points of the Pad Rear

| Pad # | r1 | r3 | r6 | r8 | Average | Std Dev | MIN | MAX |
|---|---|---|---|---|---|---|---|---|
| 1 | 3.40 | 3.59 | 2.93 | 3.02 | 3.24 | 0.31 | 2.93 | 3.59 |
| 2 | 3.08 | 3.09 | 2.62 | 2.52 | 2.83 | 0.30 | 2.52 | 3.09 |
| 3 | 3.39 | 3.48 | 2.93 | 2.87 | 3.17 | 0.31 | 2.87 | 3.48 |
| 4 | 3.07 | 3.17 | 2.66 | 2.70 | 2.90 | 0.26 | 2.66 | 3.17 |
| 5 | 3.34 | 3.54 | 2.98 | 2.97 | 3.21 | 0.28 | 2.97 | 3.54 |
| 6 | 3.12 | 3.30 | 2.70 | 2.68 | 2.95 | 0.31 | 2.68 | 3.30 |
| 7 | 3.38 | 3.55 | 2.90 | 2.99 | 3.21 | 0.31 | 2.90 | 3.55 |
| 8 | 3.06 | 3.15 | 2.70 | 2.66 | 2.89 | 0.25 | 2.66 | 3.15 |
| 9 | 3.16 | 3.48 | 3.08 | 2.89 | 3.15 | 0.25 | 2.89 | 3.48 |
| 10 | 3.08 | 3.24 | 2.80 | 2.75 | 2.97 | 0.23 | 2.75 | 3.24 |

TABLE 11
Entire Package Average, Stan Dev, Min, and Max of the Pad

| | f1 | f3 | f5 | m1 | m3 | m6 | m8 | r1 | r3 | r6 | r8 |
|---|---|---|---|---|---|---|---|---|---|---|---|
| Entire Pack | 2.80 | 2.96 | 3.25 | 3.20 | 2.66 | 3.05 | 3.15 | 3.21 | 3.36 | 2.83 | 2.81 |
| Average: | | | | | | | | | | | |
| Entire | 0.17 | 0.11 | 0.13 | 0.05 | 0.08 | 0.22 | 0.05 | 0.15 | 0.19 | 0.16 | 0.17 |
| Pack Std | | | | | | | | | | | |
| Dev: | | | | | | | | | | | |
| Entire Pack | 2.58 | 2.77 | 3.10 | 3.09 | 2.56 | 2.68 | 3.07 | 3.06 | 3.09 | 2.62 | 2.52 |
| MIN: | | | | | | | | | | | |
| Entire Pack | 3.04 | 3.11 | 3.46 | 3.25 | 2.80 | 3.33 | 3.23 | 3.40 | 3.59 | 3.08 | 3.02 |
| MAX: | | | | | | | | | | | |

### Example 3

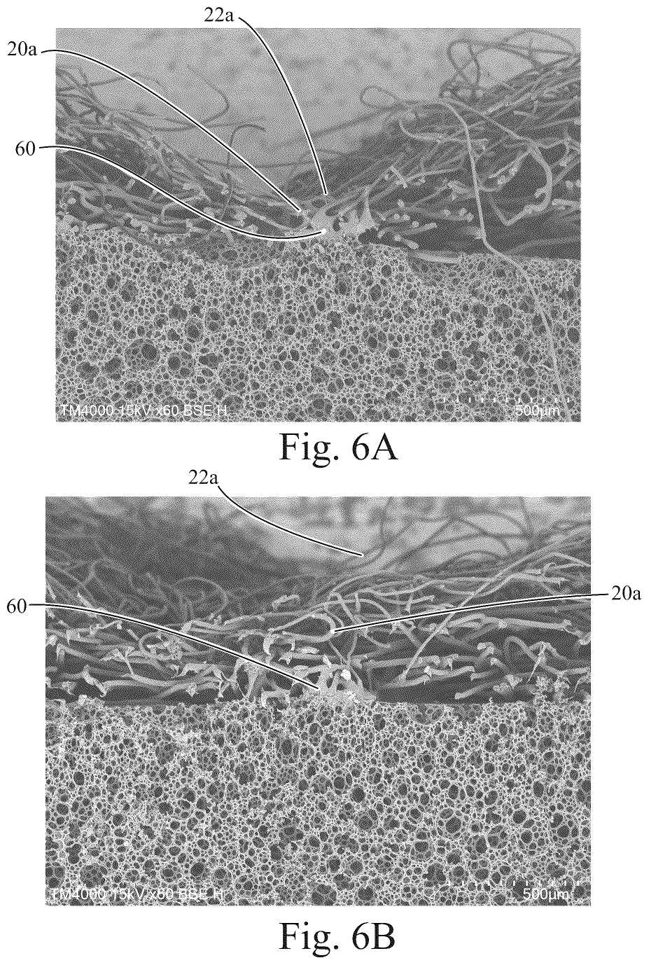

Referring to FIG. 6A and FIG. 6B, images of an absorbent article with adhesive penetrating through to the upper topsheet portion 20a and upper topsheet surface 22a (FIG. 6A) and an absorbent article where the upper topsheet portion 20a is substantially free of adhesive 60 (FIG. 6B) are shown. The absorbent article with adhesive 60 penetrating through to the upper topsheet portion and upper topsheet surface 22a created a depression in the upper topsheet portion 20a, as shown in FIG. 6A. Without being bound by theory, the depression in the upper topsheet portion 20a and upper topsheet surface 22a is believed to cause textural defects on the topsheet surface and may lower the Sdcₐ of the topsheet upper surface 22a. However, as shown in FIG. 6B, when the upper topsheet portion 20a is substantially free from adhesive 60, the upper topsheet portion 20a and upper topsheet surface 22a are not depressed, so no textural defect is created.

### Example 4

Sdcₐ was calculated for a single unstructured area of the body facing upper topsheet surface 22a of each of two absorbent articles that did not demonstrate textural defects (Sample 1 and Sample 2) and each of two absorbent articles that demonstrated textural defects (Sample 3 and Sample 4). The results are as shown in Table 12 and reported in micrometers (μm).

TABLE 12
Upper Topsheet Surface Sdcₐ

| | |
|---|---|
| Sample 1 | 750 |
| Sample 2 | 673 |
| Sample 3 | 465 |
| Sample 4 | 528 |

Sample 1 and Sample 2, which did not exhibit textural defects had Sdcₐ values that were higher than the Sdcₐ values for Sample 3 and Sample 4, which did exhibit textural defects. Thus, without being limited by theory, it is believed that absorbent articles where the upper topsheet portion and the lower topsheet portion are substantially free of adhesive have higher Sdcₐ values than the absorbent articles where the upper topsheet portion and the lower topsheet portion include adhesive.

In view of the foregoing description, the following non-limiting examples are contemplated:

A. An absorbent article comprising a liquid permeable topsheet, a liquid impervious backsheet, and an absorbent layer positioned therebetween, wherein:
the topsheet comprises:
   a fibrous nonwoven material;
   an upper topsheet surface and a lower topsheet surface, the upper topsheet surface comprising structured regions and unstructured regions; and
   a first topsheet portion adjacent the upper topsheet surface and a second topsheet portion adjacent the lower topsheet surface; and
   the absorbent layer comprises a layer of open-cell foam having an upper foam surface that is in direct facing contact with the lower topsheet surface;
wherein:
   one or more unstructured regions of the upper topsheet surface exhibit a Sdcₐ of from 530 μm to 1000 μm; and
   a portion of an adhesive penetrates into the second topsheet portion and the first topsheet portion is substantially free of adhesive.
      B. The absorbent article according to paragraph A, wherein the adhesive penetrates, by depth, from about 1% to about 70% into the topsheet.
      C. The absorbent article according to either paragraph A or paragraph B, wherein the lower topsheet surface exhibits a lower topsheet surface Sdcₜ less than 350 μm.
      D. The absorbent article according to any one of paragraphs A-C, wherein the upper topsheet surface exhibits an Sdcₜ at least 15% greater than the lower topsheet surface Sdcₜ.
      E. The absorbent article according to any one of paragraphs A-D, wherein the adhesive is a hot melt adhesive.
      F. The absorbent article according to any one of paragraphs A-E, wherein the adhesive is a spray adhesive or a fiberized adhesive.
      G. The absorbent article according to any one of paragraphs A-F, wherein the absorbent article has a basis weight of from about 100 gsm to about 600 gsm as measured according to the Absorbent Article Basis Weight Method.
      H. The absorbent article according to any one of paragraphs A-G, wherein the adhesive is a fiberized adhesive, wherein the fiberized adhesive has an average fiber thickness of from about 1 μm to about 100 μm and an average fiber length of from about 5 mm to about 50 cm.
      I. The absorbent article according to any one of paragraphs A-H, wherein the absorbent layer has a thickness of from about 1.5 mm to about 10 mm.
      J. The absorbent article according to any one of paragraphs A-I, wherein the absorbent layer has a standard deviation of thickness of from about 0.01 mm to about 0.5 mm.
      K. The absorbent article according to any one of paragraphs A-J, wherein the absorbent article has a thickness of from about 0.5 mm to about 7 mm measured according to the Thickness Test and a standard deviation of thickness of from about 0.01 mm to about 0.5 mm.
      L. The absorbent article according to any one of paragraphs A-K, wherein the absorbent layer has a first thickness in one area and a second thickness in another area, wherein the first thickness and the second thickness vary by from about 0 mm to about 4 mm.
      M. The absorbent article according to any one of paragraphs A-L, wherein the absorbent layer comprises:
a first absorbent portion comprising the upper foam surface; and
a second absorbent portion comprising a lower foam surface;
wherein the adhesive does not reach the second absorbent portion.
   N. The absorbent article according to any one of paragraphs A-M, wherein the adhesive penetrates, by depth, from about 5% to about 100% into the absorbent layer.
   O. The absorbent article according to any one of paragraphs A-N, wherein the adhesive provides an adhesive bond strength of from about 0.1 N/25 mm to about 1.0 N/25 mm as measured by the Standard Test Method for Peel Resistance of Adhesives (T-Peel Test) ASTM D-1876.
   P. The absorbent article according to any one of paragraphs A-O, wherein the topsheet has a thickness at 7 g/cm² pressure of from about 0.1 mm to about 1.3 mm, as measured according to the Thickness-Pressure Method.
   Q. The absorbent article according to any one of paragraphs A-P, further comprising a central bonding region straddling an intersection of longitudinal and lateral axes, the central bonding region having a size of at least 15 cm², within which the topsheet is bonded to the absorbent layer via a discontinuous pattern of adhesive bonds, and within which, within any identifiable first point location of an adhesive bond within the bonding region, at which the topsheet is bonded to the absorbent layer, there is a second point location at which the topsheet is bonded to the absorbent layer by adhesive, within a 10 mm radius r of the first point location.
   R. The absorbent article according to any one of paragraphs A-Q, wherein the topsheet is an unapertured topsheet.
   S. The absorbent article according to any one of paragraphs A-R, wherein the topsheet comprises hydrophilic fibers, or fibers that have been surface-treated with a surfactant.
   T. A package of two or more absorbent articles, the absorbent articles comprising a liquid permeable topsheet, a liquid impervious backsheet, and an absorbent layer positioned therebetween, wherein:
the topsheet comprises:
   a fibrous nonwoven material;
   an upper topsheet surface and a lower topsheet surface, the upper topsheet surface comprising structured regions and unstructured regions; and
   a first portion adjacent the upper topsheet surface and a second portion adjacent the lower topsheet surface;
   wherein one or more unstructured regions of the upper topsheet surface exhibit a Sdcₐ of from about 530 μm to about 1000 μm; and
   the absorbent layer comprises a layer of open-cell foam having an upper foam surface that is in direct facing contact with the lower topsheet surface; and
   wherein:
   a portion of the adhesive penetrates into the second topsheet portion and the first topsheet portion is substantially free of adhesive; and
   a portion of the absorbent layer of at least one absorbent article is from about 0.5 mm to about 4 mm greater than an area of the absorbent layer of another absorbent article in the package as measured according to the Thickness Test.
      U. The package of absorbent articles according to paragraph T, wherein the absorbent layer of at least one absorbent article has a thickness at least 15 percent greater than the absorbent layer of at least one other absorbent article.
      V. The package of absorbent articles according to either paragraph T or paragraph U, wherein a thickness of a portion of an absorbent layer of an absorbent article is from about 0.5 mm to about 4 mm greater than a thickness of a portion of an absorbent layer of another absorbent article in a different package.
      W. The absorbent article according to any one of paragraphs T-V, wherein the adhesive penetrates, by depth, from about 1% to about 70% into the topsheet.
      X The absorbent article according to any one of paragraphs T-W, wherein the lower topsheet surface exhibits a lower topsheet surface Sdcₜ less than 350 μm.
      Y. The absorbent article according to any one of paragraphs T-X, wherein the upper topsheet surface exhibits an Sdcₜ at least 15% greater than the lower topsheet surface Sdcₜ.
      Z. The absorbent article according to any one of paragraphs T-Y, wherein the adhesive is a hot melt adhesive.
      AA. The absorbent article according to any one of paragraphs T-Z, wherein the adhesive is a spray adhesive or a fiberized adhesive.
      BB. The absorbent article according to any one of paragraphs T-AA, wherein the absorbent article has a basis weight of from about 100 gsm to about 600 gsm as measured according to the Absorbent Article Basis Weight Method.
      CC. The absorbent article according to any one of paragraphs T-BB, wherein the adhesive is a fiberized adhesive, wherein the fiberized adhesive has an average fiber thickness of from about 1 μm to about 100 μm and an average fiber length of from about 5 mm to about 50 cm.
      DD. The absorbent article according to any one of paragraphs T-CC, wherein the absorbent layer has a thickness of from about 1.5 mm to about 10 mm.
      EE. The absorbent article according to any one of paragraphs T-DD, wherein the absorbent layer has a standard deviation of thickness of from about 0.01 mm to about 0.5 mm.
      FF. The absorbent article according to any one of paragraphs T-EE, wherein the absorbent article has a thickness of from about 0.5 mm to about 7 mm measured according to the Thickness Test and a standard deviation of thickness of from about 0.01 mm to about 0.5 mm.
      GG. The absorbent article according to any one of paragraphs T-FF, wherein the absorbent layer has a first thickness in one area and a second thickness in another area, wherein the first thickness and the second thickness vary by from about 0.5 mm to about 4 mm as determined according to the Thickness Test.
      HH. The absorbent article according to any one of paragraphs T-GG, wherein the absorbent layer comprises:
a first absorbent portion comprising the upper foam surface; and
a second absorbent portion comprising a lower foam surface;
wherein the adhesive does not reach the second absorbent portion.
   II. The absorbent article according to any one of paragraphs T-HH, wherein the adhesive penetrates, by depth, from about 5% to about 100% into the absorbent layer.
   JJ. The absorbent article according to any one of paragraphs T-II, wherein the adhesive provides an adhesive bond strength of from about 0.1 N/25 mm to about 1.0 N/25 mm as measured by the Standard Test Method for Peel Resistance of Adhesives (T-Peel Test) ASTM D-1876.
   KK. The absorbent article according to any one of paragraphs T-JJ, wherein the topsheet has a thickness at 7 g/cm² pressure of from about 0.1 mm to about 1.3 mm, as measured according to the Thickness-Pressure Method.
   LL. The absorbent article according to any one of paragraphs T-KK, further comprising a central bonding region straddling the an intersection of longitudinal and lateral axes, the central bonding region having a size of at least 15 cm², within which the topsheet is bonded to the absorbent layer via a discontinuous pattern of adhesive bonds, and within which, within any identifiable first point location of an adhesive bond within the bonding region, at which the topsheet is bonded to the absorbent layer, there is a second point location at which the topsheet is bonded to the absorbent layer by adhesive, within a 10 mm radius r of the first point location.
   MM. The absorbent article according to any one of paragraphs T-LL, wherein the topsheet is an unapertured topsheet.
   NN. The absorbent article according to any one of paragraphs T-MM, wherein the topsheet comprises hydrophilic fibers, or fibers that have been surface-treated with a surfactant.
   OO. A method of producing an absorbent article, the method comprising:
obtaining a liquid permeable topsheet, a liquid impervious backsheet, and an absorbent layer, wherein:
   the topsheet comprises:
      a fibrous nonwoven material;
      an upper topsheet surface and a lower topsheet surface; and
      a first portion adjacent the upper topsheet surface and a second portion adjacent the lower topsheet surface;
      a Sdcₐ of from 530 μm to 1000 μm when the topsheet is bonded to the absorbent layer with an adhesive; and
      the absorbent layer comprises a layer of open-cell foam having an upper foam surface that is in direct facing contact with the lower topsheet surface;
      applying adhesive to either the topsheet, the absorbent layer, or a combination thereof; and
      compressing the topsheet and the absorbent layer with a pressure, wherein the pressure is selected so that the topsheet is at least partially attached to the absorbent layer using an adhesive, wherein the adhesive does not reach the first topsheet portion.
         PP. The absorbent article according to paragraph OO, wherein the adhesive penetrates, by depth, from about 1% to about 70% into the topsheet.
         QQ. The method according to either paragraph OO or paragraph PP, wherein the lower topsheet surface exhibits a lower topsheet surface Sdcₜ less than about 350 μm.
         RR. The method according to any one of paragraphs OO-QQ, wherein the upper topsheet surface exhibits an Sdcₜ at least 15% greater than the lower topsheet surface Sdcₜ.
         SS The method according to any one of paragraphs OO-RR, wherein the adhesive is a hot melt adhesive.
         TT. The method according to any one of paragraphs OO-SS, wherein the adhesive is a spray adhesive or a fiberized adhesive.
         UU. The method according to any one of paragraphs OO-TT, wherein the absorbent article has a basis weight of from about 100 gsm to about 600 gsm as measured according to the Absorbent Article Basis Weight Method.
         VV. The method according to any one of paragraphs OO-UU, wherein the adhesive is a fiberized adhesive, wherein the fiberized adhesive has an average fiber thickness of from about 1 μm to about 100 μm and an average fiber length of from about 5 mm to 50 about cm.
         WW. The method according to any one of paragraphs OO-VV, wherein the absorbent layer has a thickness of from about 1.5 mm to about 10 mm.
         XX. The method according to any one of paragraphs OO-WW, wherein the absorbent layer has a standard deviation of thickness of from about 0.01 mm to about 0.5 mm.
         YY. The method according to any one of paragraphs OO-XX, wherein the absorbent article has a thickness of from about 0.5 mm to about 7 mm measured according to the Thickness Test and a standard deviation of thickness of from about 0.01 mm to about 0.5 mm.
         ZZ. The method according to any one of paragraphs OO-YY, wherein the absorbent layer has a first thickness in one area and a second thickness in another area, wherein the first thickness and the second thickness vary by from about 0.5 mm to about 4 mm as determined by the Thickness Test.
         AAA. The method according to any one of paragraphs OO-ZZ, wherein the absorbent layer comprises:
a first absorbent portion comprising the upper foam surface; and
a second absorbent portion comprising a lower foam surface;
wherein the adhesive does not reach the second absorbent portion.
   BBB. The method according to any one of paragraphs OO-AAA, wherein the adhesive penetrates, by depth, from about 5% to about 100% into the absorbent layer.
   CCC. The method according to any one of paragraphs OO-BBB, wherein the adhesive provides an adhesive bond strength of from about 0.1 N/25 mm to about 1.0 N/25 mm as measured by the Standard Test Method for Peel Resistance of Adhesives (T-Peel Test) ASTM D-1876.
   DDD. The method according to any one of paragraphs OO-CCC, wherein the topsheet has a thickness at 7 g/cm² pressure of from about 0.1 mm to about 1.3 mm, as measured according to the Thickness-Pressure Method.
   EEE. The method according to any one of paragraphs OO-DDD, further comprising a central bonding region straddling the an intersection of longitudinal and lateral axes, the central bonding region having a size of at least 15 cm², within which the topsheet is bonded to the absorbent layer via a discontinuous pattern of adhesive bonds, and within which, within any identifiable first point location of an adhesive bond within the bonding region, at which the topsheet is bonded to the absorbent layer, there is a second point location at which the topsheet is bonded to the absorbent layer by adhesive, within a 10 mm radius r of the first point location.
   FFF. The method according to any one of paragraphs OO-EEE, wherein the topsheet is an unapertured topsheet.
   GGG. The method according to any one of paragraphs OO-FFF, wherein the topsheet comprises hydrophilic fibers, or fibers that have been surface-treated with a surfactant.
   HHH. The method according to any one of paragraphs OO-FFF, wherein the pressure is varied based on a variation in thickness of the absorbent layer.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular configurations of the present disclosure have been illustrated and described, it would be obvious to those skilled in the art that various other changes and modifications can be made without departing from the spirit and scope of the invention. It is therefore intended to cover in the appended claims all such changes and modifications that are within the scope of the present disclosure.

What is claimed is:

1. An absorbent article comprising a liquid permeable topsheet, a liquid impervious backsheet, and an absorbent layer positioned therebetween, wherein:
the topsheet comprises:
a fibrous nonwoven material;
an upper topsheet surface and a lower topsheet surface, the upper topsheet surface comprising structured regions and unstructured regions; and
a first topsheet portion adjacent the upper topsheet surface and a second topsheet portion adjacent the lower topsheet surface; and
the absorbent layer comprises a layer of open-cell foam having an upper foam surface that is in direct facing contact with the lower topsheet surface;
wherein:
one or more unstructured regions of the upper topsheet surface exhibit a Sdcₐ of from 530 μm to 1000 μm, preferably from 540 μm to 1000 μm, and more preferably from 550 μm to 1000 μm; and
a portion of an adhesive penetrates into the second topsheet portion and the first topsheet portion is substantially free of adhesive.

2. The absorbent article of claim 1, wherein the adhesive penetrates, by depth, from 1% to 70% into the topsheet.

3. The absorbent article of claim 1, wherein the lower topsheet surface exhibits a lower topsheet surface Sdcₜ less than 350 μm.

4. The absorbent article of claim 1, wherein the upper topsheet surface exhibits an Sdcₜ at least 15% greater than the lower topsheet surface Sdcₜ.

5. The absorbent article of claim 1, wherein the adhesive is a spray adhesive or a fiberized adhesive.

6. The absorbent article of claim 1, wherein the absorbent layer has a thickness of from 1.5 mm to 10 mm and a standard deviation of thickness of from 0.01 mm to 0.5 mm.

7. The absorbent article of claim 1, wherein the absorbent article has a thickness of from 0.5 mm to 7 mm measured according to the Thickness Test and a standard deviation of from 0.01 mm to 0.5 mm.

8. The absorbent article of claim 1, wherein the absorbent layer comprises:
a first absorbent portion comprising the upper foam surface; and
a second absorbent portion comprising a lower foam surface;
wherein the adhesive does not reach the second absorbent portion.

9. The absorbent article of claim 1, wherein the adhesive penetrates, by depth, from 5% to 100% into the absorbent layer.

10. The absorbent article of claim 1, wherein the adhesive provides an adhesive bond strength of from 0.1 N/25 mm to 1.0 N/25 mm, as measured by the Standard Test Method for Peel Resistance of Adhesives (T-Peel Test) ASTM D-1876.

11. The absorbent article of claim 1, wherein the topsheet is an unapertured topsheet.

12. A package of two or more absorbent articles of claim 1,
wherein:
the absorbent layer of at least one absorbent article is from 0.5 mm to 4 mm greater, preferably from 1 mm to 4 mm greater, more preferably from 1.5 mm to 4 mm greater than an absorbent layer of another absorbent article in the package as determined according to the Thickness Test.

13. The package of absorbent articles of claim 12, wherein the absorbent layer of at least one absorbent article has a thickness at least 15% greater, preferably at least 18% greater, more preferably at least 20% greater than the absorbent layer of at least one other absorbent article.

14. The package of absorbent articles of claim 12, wherein a thickness of a portion of an absorbent layer of an absorbent article is from 0.5 mm to 4 mm greater, preferably from 1 mm to 4 mm greater, more preferably from 1.5 mm to 4 mm greater than a thickness of a portion of an absorbent layer of another absorbent article in a different package.

15. A method of producing the absorbent article of claim 1, the method comprising:
obtaining a liquid permeable topsheet, a liquid impervious backsheet, and an absorbent layer, wherein:
the topsheet comprises:
a fibrous nonwoven material;
an upper topsheet surface and a lower topsheet surface; and
a first portion adjacent the upper topsheet surface and a second portion adjacent the lower topsheet surface;
wherein the topsheet exhibits a Sdcₜ of from 350 μm to 1000 μm, preferably from 375 μm to 1000 μm, more preferably from 420 to 1000 μm, before the topsheet is bonded to the absorbent layer and a Sdcₐ of from 530 μm to 1000 μm, preferably from 540 μm to 1000 μm, and more preferably from 550 μm to 1000 μm when the topsheet is bonded to the absorbent layer with an adhesive; and
the absorbent layer comprises a layer of open-cell foam having an upper foam surface that is in direct facing contact with the lower topsheet surface;
applying adhesive to either the topsheet or the absorbent layer; and
compressing the topsheet and the absorbent layer with a pressure, wherein the pressure is selected so that the topsheet is at least partially attached to the absorbent layer using an adhesive, wherein the adhesive does not reach the first topsheet portion.
